(19) United States
(12) Patent Application Publication
(10) Pub. No.: US 2007/0043084 A1
(43) Pub. Date: Feb. 22, 2007

(54) 1,2-Diarylimidazoles useful as inhibitors of cox

(75) Inventors: Fumie Takahashi, Tokyo (JP); Tadashi Terasaka, Tokyo (JP); Masataka Morita, Tokyo (JP); Nobukiyo Konishi, Tokyo (JP); Katsuya Nakamura, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Astellas Pharma Inc., Tokyo (JP)

(21) Appl. No.: 10/555,656

(22) PCT Filed: Apr. 26, 2004
(86) PCT No.: PCT/JP04/05987

§ 371 Date: Nov. 4, 2005

(30) Foreign Application Priority Data
May 8, 2003 (AU) 2003902208
Jul. 24, 2003 (AU) 2003903861
Aug. 1, 2003 (AU) 2003904068

(51) Int. Cl. A61K 31/444; A61K 31/4439; C07D 403/04

(52) U.S. Cl. 514/332; 514/341; 546/256; 546/272.7

(57) ABSTRACT

A compound of the formula (1): wherein R¹ is cyano, and the like; R² is hydroxy, and the like; R³ is (lower)alkoxy, and the like; X and Y are each CH or N; or pharmaceutically acceptable salts thereof, which are useful as a medicament.

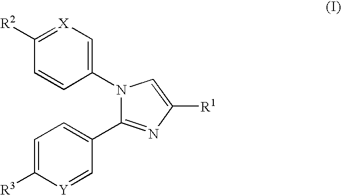

### TECHNICAL FIELD

This invention relates to imidazole compounds and pharmaceutically acceptable salts thereof having pharmacological activity.

Moreover, this invention relates to medicament or pharmaceutical composition comprising the above mentioned imidazole compounds or pharmaceutically acceptable salts thereof as an active ingredient.

### BACKGROUND ART

Some imidazole derivatives having anti-inflammatory and/or analgesic activities have been known, for example, WO 96/03388. However, all of compounds disclosed in this document are substituted by sulfonyl group on imidazole ring. Further, the compounds disclosed in WO 96/03388 selectively inhibit cyclooxygenase-II (COX-II) over cyclooxygenase-I (COX-I).

### DISCLOSURE OF THE INVENTION

As a result of studies on the synthesis of imidazole compounds and their pharmacological activity, the inventors of this invention have found that the imidazole compounds of this invention have superior activity of inhibiting COX (especially, COX-I inhibiting activity). Therefore, this invention relates to imidazole compounds, which have pharmaceutical activity such as COX inhibiting activity, and to a medicament and a pharmaceutical composition containing the imidazole compound.

Accordingly, one object of this invention is to provide the imidazole compounds, which have a COX inhibiting activity.

Another object of this invention is to provide a method for treatment and/or prevention and the imidazole compounds for use in the treatment and/or prevention of the disease associated with COX.

A further object of this invention is to provide a use of the imidazole compounds for manufacturing a medicament for treating or preventing the diseases and to an analgesic agent comprising the imidazole compounds which is usable for treating and/or preventing pains.

A further object of this invention is to provide the commercial package comprising the pharmaceutical composition containing the new compound.

The imidazole compounds of this invention can be represented by the following general formula (I): 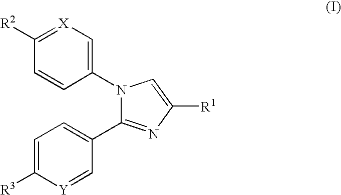
[wherein
R¹ is (lower)alkyl, halogen-substituted (lower)alkyl, hydroxy-substituted (lower)alkyl, cycloalkyl, carbamoyl, N-[(lower)alkyl]carbamoyl, N,N-di[(lower)alkyl]carbamoyl, formyl, (lower)alkanoyl, carboxy, [(lower)alkoxy]carbonyl, cyano, cycloalkylcarbonyl or heterocycliccarbonyl;
R² is halogen, cyano, hydroxy, (lower)alkoxy, aryl[(lower)alkyl]oxy, [(lower)alkoxy]carbonyl, carbamoyl, formyloxy, (lower)alkanoyloxy, [(lower)alkyl]sulfonyloxy, [halogen-substituted (lower)alkyl]sulfonyloxy or carboxy;
R³ is (lower)alkoxy, hydroxy, amino, [(lower)alkyl]amino, or di[(lower)alkyl]amino;
X and Y are each CH or N]
   or pharmaceutically acceptable salts thereof.

In the above and subsequent description of the present specification, suitable examples of the various definitions to be included within the scope of the invention are explained in detail in the following.

The term “lower” is intended to mean a group having 1 to 6 carbon atom(s), unless otherwise provided.

Accordingly, the “(lower)alkyl” means a straight or branched chain aliphatic hydrocarbon, such as methyl, ethyl, propyl, isopropyl, butyl, isobutyl, tert-butyl, pentyl, hexyl, and the like, and it is preferably (C1-C4)alkyl, more preferably (C1-C2)alkyl, most preferably methyl.

The “halogen” may include a fluorine atom, a chlorine atom, a bromine atom and an iodine atom, and is preferably a fluorine atom or a chlorine atom, more preferably a fluorine atom.

The “halogen-substituted (lower)alkyl” means the above lower alkyl substituted by the above halogen atom(s), such as fluoromethyl, chloromethyl, difluoromethyl, dichloromethyl, dibromomethyl, trifluoromethyl, trichloromethyl, fluoroethyl, chloroethyl, 2.2,2-trifluoroethyl, 2,2,2-trichloroethyl, 2,2,3,3,3-pentafluoroethyl, fluoropropyl, fluorobutyl, fluorohexyl, and the like, and it is preferably halogen-substituted (C1-C4)alkyl, more preferably halogen-substituted (C1-C2)alkyl, more preferably fluorine-substituted (C1-C2)alkyl, more preferably fluorine-substituted methyl, most preferably difluoromethyl or trifluoromethyl.

The “hydroxy-substituted (lower)alkyl” means the above (lower)alkyl substituted by a OH group, such as hydroxymethyl, hydroxyethyl, hydroxypropyl, 1-hydroxyisopropyl, 2-hydroxyisopropyl, hydroxybutyl, hydroxyisobutyl, hydroxy-tert-butyl, hydroxyhexyl, and the like, and it is preferably hydroxy-substituted (C1-C4)alkyl, more preferably hydroxy-substituted (C1-C2)alkyl, most preferably hydroxymethyl.

The “cycloalkyl” means (C3-C10)cycloalkyl group, such as cyclopropyl, cyclobutyl, cyclopentyl, cyclohexyl, cycloheptyl, norbornyl, adamantyl, and the like, and it is preferably (C3-C6) cycloalkyl, more preferably (C3-C5) cycloalkyl, most preferably cyclopropyl.

Therefore, the “cycloalkylcarbonyl” means carbonyl group substituted by the above cycloalkyl group, such as cyclopropylcarbonyl, cyclobutylcarbonyl, cyclopentylcarbonyl, cyclohexylcarbonyl, cycloheptylcarbonyl, norbornylcarbonyl, adamantylcarbonyl, and the like, and it is preferably [(C3-C6) cycloalkyl]carbonyl, more preferably [(C3-C5)cycloalkyl]carbonyl.

The “N-[(lower)alkyl]carbamoyl” means a carbamoyl group substituted by one (lower)alkyl group mentioned above on nitrogen atom, such as methylcarbamoyl, ethylcarbamoyl, propylcarbamoyl, isopropylcarbamoyl, butylcarbamoyl, isobutylcarbamoyl, tert-butylcarbamoyl, pentylcarbamoyl, hexylcarbamoyl, and the like, and it is preferably N-[(C1-C4)alkyl]carbamoyl, more preferably N-[(C1-C2)alkyl]carbamoyl.

The “N,N-di[(lower)alkyl]carbamoyl” means a carbamoyl group substituted by the same or different two (lower)alkyl groups mentioned above on nitrogen atom, such as dimethylcarbamoyl, diethylcarbamoyl, dipropylcarbamoyl, diisopropylcarbamoyl, dibutylcarbamoyl, diisobutylcarbamoyl, dipentylcarbamoyl, dihexylcarbamoyl, ethylmethylcarbamoyl, methylpropylcarbamoyl, butylmethylcarbamoyl, ethylpropylcarbamoyl, butylethylcarbamoyl, and the like, and it is preferably di[(C1-C4)alkyl]carbamoyl, more preferably di[(C1-C2)alkyl]carbamoyl.

The “(lower)alkanoyl” means carbonyl group which is substituted by the above (lower)alkyl groups, such as acetyl, propionyl (ethylcarbonyl), butyryl, isobutyryl (isopropylcarbonyl), pivaloyl, valeryl, isovaleryl, hexanoyl, and the like, and it is preferably (C2-C5)alkanoyl, more preferably (C2-C4)alkanoyl.

Therefore, the “(lower)alkanoyloxy” may be exemplified by acetyloxy, propionyloxy (ethylcarbonyloxy), butyryloxy, isobutyryloxy (isopropylcarbonyloxy), pivaloyloxy, valeryoxyl, isovaleryloxy, hexanoyloxy, and the like, and it is preferably (C2-C5)alkanoyloxy, more preferably (C2-C4)alkanoyloxy.

The “(lower)alkoxy” means a straight or branched chain aliphatic hydrocarbon oxy group, such as methoxy, ethoxy, propoxy, isopropoxy, butoxy, isobutoxy, tert-butoxy, pentoxy, hexoxy, and the like, and it is preferably (C1-C4)alkoxy, more preferably (C1-C2)alkoxy, most preferably methoxy.

Therefore, the “[(lower)alkoxy]carbonyl” means a —CO₂-[(lower)alkyl]group, such as methoxycarbonyl, ethoxycarbonyl, propoxycarbonyl, isopropoxycarbonyl, butoxycarbonyl, isobutoxycarbonyl, tert-butoxycarbonyl, pentoxycarbonyl, hexoxycarbonyl, and the like, and it is preferably [(C1-C4)alkoxy]carbonyl, more preferably ethoxycarbonyl.

The “heterocycle” means 5- or 6-membered saturated heterocyclic group which contains at least one hetero atom such as nitrogen, oxygen, sulfur atom. The “heterocycle” may include 5-membered heterocyclic group such as pyrrolidinyl, imidazolidinyl, pyrazolidinyl, tetrahydrothiophenyl, tetrahydrofuranyl, oxazolidinyl, isoxazolidinyl, thiazolidinyl, isothiazolidinyl, or the like; and 6-membered heterocyclic group such as piperidinyl, piperazinyl, morpholinyl, thiomorpholinyl, or the like.

Therefore, the “heterocycliccarbonyl” may be exemplified pyrrolidinylcarbonyl, imidazolidinylcarbonyl, pyrazolidinylcarbonyl, tetrahydrothiophenylcarbonyl, tetrahydrofuranylcarbonyl, oxazolidinylcarbonyl, isoxazolidinylcarbonyl, thiazolidinylcarbonyl as 5-membered heterocycliccarbonyl group; and piperidinylcarbonyl, piperazinylcarbonyl, morpholinylcarbonyl, thiomorpholinylcarbonyl as 6-membered heterocycliccarbonyl group. This group is preferably (heterocyclic containing nitrogen atom)carbonyl or 6-membered heterocycliccarbonyl, more preferably piperidinylcarbonyl.

The “aryl[(lower)alkyl]oxy,” means the above mentioned (lower)alkoxy group which is substituted with aryl group, such as benzyloxy, naphtylmethyloxy, indenylmethyloxy, phenetyl, naphtylethyl, phenylpropyl, phenylbutyl, phenylhexyl, and the like, and it is preferably aryl[(C1-C2)alkyl]oxy, more preferably arylmethoxy, most preferably benzyloxy.

The “[(lower)alkyl]sulfonyl” means a sulfonyl group substituted with (lower)alkyl group mentioned above, such as mathanesulfonyl, ethanesulfonyl, isopropanesulfonyl, tert-butanesulfonyl, and the like, and it is preferably (C1-C4)alkanesulfonyl, more preferably (C1-C2)alkanesulfonyl, most preferably methanesulfonyl.

Therefore, the “[(lower)alkyl]sulfonyloxy” may be exemplified by mathanesulfonyloxy, ethanesulfonyloxy, isopropanesulfonyloxy, tert-butanesulfonyloxy, and the like, and it is preferably (C1-C4)alkanesulfonyloxy, more preferably (C1-C2)alkanesulfonyloxy, most preferably methanesulfonyloxy.

The “[halogen-substituted (lower)alkyl]sulfonyl” meansasulfonyl group substituted with halogen-substituted (lower)alkyl mentioned above, such as trifluoromathanesulfonyl, and the like, and it is preferably [halogen-substituted (C1-C4)alkyl]sulfonyl, more preferably [halogen-substituted (C1-C2)alkyl]sulfonyl, most preferably trifluoromathanesulfonyl.

Therefore, the “[halogen-substituted (lower)alkyl]sulfonyloxy” may be exemplified by trifluoromathanesulfonyloxy, and the like, and it is preferably [halogen-substituted (C1-C4)alkyl]sulfonyloxy, more preferably [halogen-substituted (C1-C2)alkyl]sulfonyloxy, most preferably trifluoromathanesulfonyloxy.

The “[(lower)alkyl]amino” means a amino group substituted by one lower alkyl group mentioned above, such as methylamino, ethylamino, propylamino, isopropylamino, butylamino, isobutylamino, tert-butylamino, pentylamino, hexylamino, and the like, and it is preferably [(C1-C4)alkyl]amino, more preferably [(C1-C2)alkyl]amino.

The “di[(lower)alkyl]amino” means a amino group substituted by the same or different two (lower)alkyl groups mentioned above, such as dimethylamino, diethylamino, dipropylamino, diisopropylamino, dibutylamino, diisobutylamino, dipentylamino, dihexylamino, ethylmethylamino, methylpropylamino, butylmethylamino, ethylpropylamino, butylethylamino, and the like, and it is preferably di[(C1-C4)alkyl]amino, more preferably di[(C1-C2)alkyl]amino.

The combination of X and Y is X and Y are each CH, X is N and Y is CH, X is CH and Y is N, X and Y are each N, preferably both of X and Y are CH, X is N and Y is CH, or X is CH and Y is N, and any of these three combination are preferable.

The compounds of formula (I) may contain one or more asymmetric centers and thus they can exist as enantiomers or diastereoisomers. This invention includes both mixtures and separate individual isomers.

The compounds of the formula (I) may also exist in tautomeric forms and the invention includes both mixtures and separate individual tautomers.

The compounds of the formula (I) and its salts can be in a form of a solvate, which is included within the scope of the present invention. The solvate preferably include a hydrate.

Also included in the scope of invention are radiolabelled derivatives of compounds of formula (I) which are suitable for biological studies.

The imidazole compounds of this invention can be converted to salt according to a conventional method. Suitable salts of the compounds (I) are pharmaceutically acceptable conventional non-toxic salts and include a metal salt such as an alkali metal salt (e.g., sodium salt, potassium salt, or the like.) and an alkaline earth metal salt (e.g., calcium salt, magnesium salt, or the like.), an ammonium salt, an organic base salt (e.g., trimethylamine salt, triethylamine salt, pyridine salt, picoline salt, dicyclohexylamine salt, or the like.), an organic acid salt (e.g., acetate, maleate, tartrate, methanesulfonate, benzenesulfonate, formate, toluenesulfonate, trifluoroacetate, or the like.), an inorganic acid salt (e.g., hydrochloride, hydrobromide, sulfate, phosphate, or the like.), a salt with an amino acid (e.g., arginate, aspartate, glutamate, or the like.), or the like.

The imidazole compound (I) may preferably include

wherein

R¹ is (lower)alkyl, halogen-substituted (lower)alkyl, cycloalkyl, N,N-di[(lower)alkyl]carbamoyl, (lower)alkanoyl, or cyano;
R² is halogen, cyano, hydroxy, or lower alkoxy;
R³ is lower alkoxy;
X and Y are each CH, X is N and Y is CH, or X is CH and Y is N.

In the each definition of the compound formula (I), preferably.
(1) R¹ is (lower)alkyl, halogen-substituted (lower)alkyl, cycloalkyl, carbamoyl, N,N-di[(lower)alkyl]carbamoyl, (lower)alkanoyl or cyano,
(2) R¹ is (lower)alkyl, halogen-substituted (lower)alkyl, cycloalkyl,
(3) R¹ is (C1-C4)alkyl, halogen-substituted (C1-C4)alkyl or (C3-C6)cycloalkyl,
(4) R¹ is (C1-C2)alkyl, halogen-substituted (C1-C2)alkyl or (C3-C5)cycloalkyl,
(5) R¹ is carbamoyl or N,N-di[(C1-C4)alkyl]carbamoyl,
(6) R¹ is carbamoyl or N,N-di[(C1-C2)alkyl]carbamoyl,
(7) R¹ is (C2-C4)alkanoyl or cyano,
(8) R² is halogen, cyano, hydroxy, (lower)alkoxy, aryl[(lower)alkyl]oxy, [(lower)alkoxy]carbonyl, carbamoyl or [halogen-substituted (lower)alkyl]sulfonyloxy,
(9) R² is halogen, cyano, hydroxy, (C1-C4)alkoxy, arylmethoxy, [(C1-C4)alkoxy]carbonyl, carbamoyl or [halogen-substituted (C1-C4)alkyl]sulfonyloxy,
(10) R² is halogen, cyano, hydroxy or (C1-C2)alkoxy,
(11) R² is hydroxy or (C1-C2)alkoxy,
(12) R³ is (lower)alkoxy or hydroxy,
(13) R³ is (C1-C4)alkoxy,
(14) R³ is (C1-C2)alkoxy,
(15) X and Y are each CH,
(16) X is N and Y is CH,
(17) X is CH and Y is N.
(18) X and Y are each N.

The compound of the formula (I) of the present invention can be prepared according to the following process.

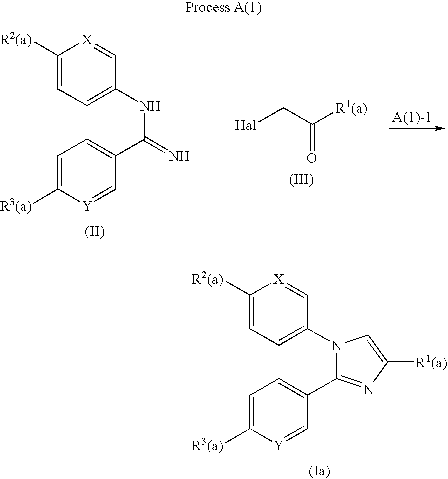

In the above formulae, X and Y represent the same meanings as defined above. R¹(a), R²(a) and R³(a) represent the group in the definition of R¹, R² and R³, respectively, which do not influence this process. Specifically, R¹(a) represents (lower)alkyl, halogen-substituted (lower)alkyl, cycloalkyl, N,N-di[(lower)alkyl]carbamoyl, formyl, (lower)alkanoyl, [(lower)alkoxy]carbonyl, cyano or cycloalkylcarbonyl; R²(a) represents halogen, cyano, (lower)alkoxy, aryl[(lower)alkyl]oxy, [(lower)alkoxy]carbonyl, formyloxy, (lower)alkanoyloxy, [(lower)alkyl]sulfonyloxy or [halogen-substituted (lower)alkyl]sulfonyloxy; R³(a) represents lower alkoxy. “Hal” represents halogen atom, especially, chlorine or bromine atom.

Process A(1) is the process for preparing the compound (Ia), which corresponds to compound (I) in which R¹ to R³ are not reactive groups.

This process is carried out by reacting compound (II) and compound (III) in the presence of base to form imidazole ring.

Compound (II) may be purchased if it is commercial, or synthesized according to Process B mentioned after or other general methods from commercial compounds. Compound (III) may be purchased if it is commercial, or synthesized according to general methods from commercial compounds, because compound (III) as starting compound for synthesis of compound (Ia) have comparatively simple structure.

The solvent employable in this process is not particularly limited so long as it is inactive in this reaction and may include alcohols such as methanol, ethanol, 2-propanol; ethers such as diisopropyl ether, tatrahydrofuran, dioxane; and mixed solvent thereof.

The base employable in this process for making basic condition is not particularly limited so long as it accelerates this reaction and may include alkali metal hydrogencarbonates such as lithium hydrogencarbonate, sodium hydrogencarbonate and potassium hydrogencarbonate; alkali metal carbonates such as lithium carbonate, sodium carbonate and potassium carbonate; alkaline earth metal carbonates such as magnesium carbonate and calcium carbonate; alkali metal hydroxides such as lithium hydroxide, sodium hydroxide and potassium hydroxide, preferably alkali metal hydrogencarbonates, especially sodium hydrogencarbonate.

The reaction temperature varies depending on the starting material, the solvent, etc., but it is usually from 50° C. to 150° C., preferably from 60° C. to 100° C. or reflux condition.

The reaction time varies depending on the starting material, the solvent, the reaction temperature, etc., but it is usually from 1 hr to 1 day, preferably from 2 hrs to 12 hrs.

After the reaction, the reaction mixture is cooled to room temperature and evaporated in vacuo, then added water and extracted with organic solvent immiscible with water such as ethyl acetate. The organic layer is washed with water or the like, dried over anhydrous magnesium sulfate or anhydrous sodium sulfate, evaporated in vacuo, and the desired compound is purified by the conventional method such as silica gel column chromatography, recrystallization, or the like.

According to the starting material, the heterocyclic ring may be formed but not to form imidazole ring sometimes. In such case, the dehydration process is needed to form imidazole ring.

The dehydration process is carried out in the hot and acidic condition.

The solvent employable in this process is not particularly limited, but acid such as acetic acid, sulfuric acid or the like may be used as solvent.

The reaction temperature varies depending on the starting material, the solvent, etc. but it is usually from 50° C. to 200° C. preferably from 80° C. to 150° C.

The reaction time varies depending on the starting material, the solvent, the reaction temperature, etc., but it is usually from 30 min to 5 hrs, preferably from 1 hr to 3 hrs.

After the reaction, the mixture is poured into basic water, and extracted with organic solvent in soluble with water such as ethylacetate. The organic layer is washed with water or the like, dried over anhydrous magnesium sulfate or anhydrous sodium sulfate, evaporated in vacuo, and the desired compound is purified by the conventional method such as silica gel column chromatography, recrystallization, or the like.

Compound (Ia) can also be synthesized according to the following process.

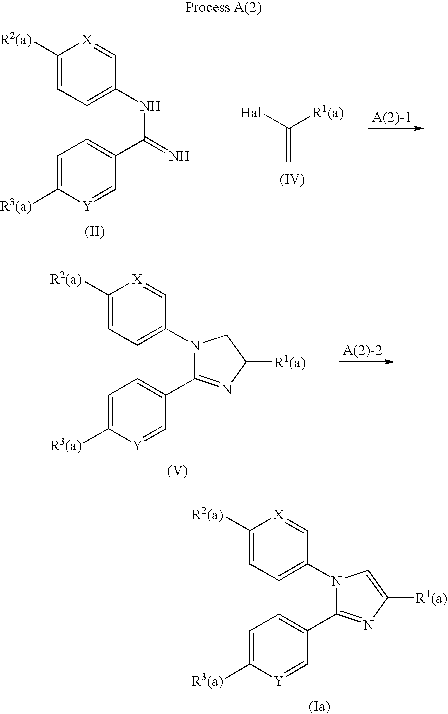

In the above formulae, R¹(a), R²(a), R³(a), X, Y and Hal represent the same meanings as defined above.

Process A(2) is the process for preparing the compound (Ia), which corresponds to compound (I) in which R¹ to R³ are not reactive groups.

In this process, first, compound (II) is condensed to compound (IV) for synthesis of compound (V) (Process A(2)-1).

Process A(2)-1 can be carried out under in the presence of Hunig's base (N,N-diisopropylethylamine).

Compound (IV) may be purchased if it is commercial, or synthesized according to general methods from commercial compounds, because compound (IV) have comparatively simple structure.

The solvent employable in this process is not particularly limited so long as it is inactive in this reaction and may include ethers such as diisopropyl ether, tatrahydrofuran, dioxane, preferably tetrahydrofuran.

The reaction temperature varies depending on the starting material, the solvent, etc., but it is usually from 50° C. to 200° C., preferably from 50° C. to 120° C. or reflux condition.

The reaction time varies depending on the starting material, the solvent, the reaction temperature, etc., but it is usually from 1 hr to 2 days, preferably 1 hr to 5 hrs or over night.

If the reaction does not proceed adequately, additional compound (IV) may be added.

After the reaction, the desired compound (V) is collected from the reaction mixture according to a conventional method. For example, after cooled to room temperature and evaporated in vacuo, the reaction mixture is poured into water and extracted with organic solvent immiscible with water such as ethyl acetate. The organic solvent is washed with water or the like, dried over anhydrous magnesium sulfate or sodium sulfate, evaporated in vacuo, and the desired compound is purified by the conventional method such as silica gel column chromatography, recrystallization, etc.

Process A(2)-2 is the oxidation process to form imidazole ring in the presence of catalyst.

The oxidative catalyst employable in this process is not particularly limited so long as it can catalyze the reaction from 4,5-dihydro-imidazole derivative (V) to imidazole derivative and may include manganese(IV) oxide (MnO₂).

The solvent employable in this process is not particularly limited so long as it is inactive in this reaction and may include amides such as N,N-dimethylformamide, dimethylacetamide, hexamethylphosphoric triamide; aromatic hydrocarbon such as benzene, toluene; or the like.

The reaction temperature varies depending on the starting material, the solvent, etc., but it is usually from 50° C. to 200° C., preferably from 80° C. to 120° C. or reflux condition.

The reaction time varies depending on the starting material, the solvent, the reaction temperature, etc., but it is usually from 1 hr to 24 hrs, preferably 2 hrs to 12 hrs.

If the reaction does not proceed adequately, additional catalyst may be added.

After the reaction, the mixture is cooled to room temperature and filtered to remove catalyst. The organic fraction is concentrated in vacuo, or poured into basic water, and extracted with organic solvent insoluble with water such as ethyl acetate. The organic layer is washed with water or the like, dried over anhydrous magnesium sulfate or anhydrous sodium sulfate, and evaporated in vacuo. The desired compound is purified by the conventional method such as silica gel column chromatography, recrystallization, or the like.

Compound (Ia) can be transformed into compound (I) by functional group trans formation, which is obvious to the person skilled in the organic chemistry. For example, such reactions are illustrated as following.

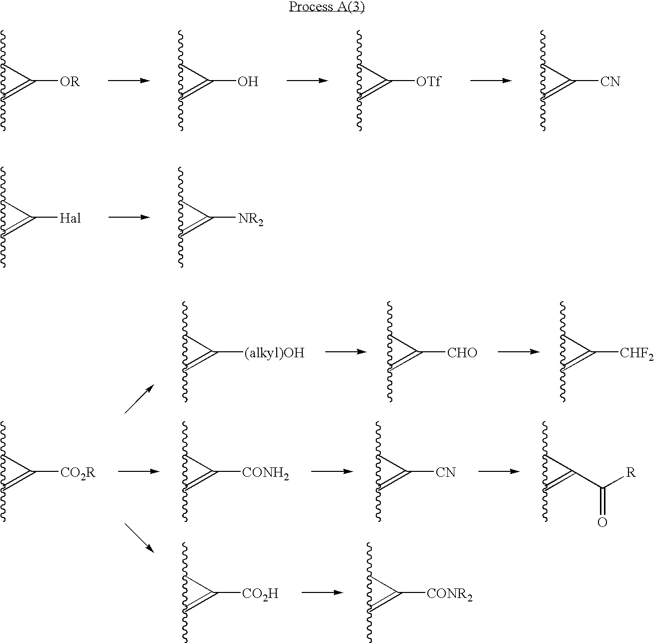

In the above formulae, R represents H, (lower)alkyl or aryl[(lower)alkyl]group, which is not specified. “Tf” represents trifluoromethanesulfonyl as protective group.

4,5-Dihydoroimidazole compound (V) can be transformed into compound (IX) by the above mentioned functional group trans formation.
Process A(4)

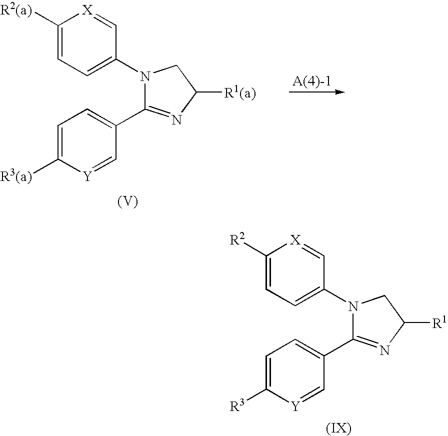

Compound (IX) or pharmaceutically acceptable salts thereof also has an inhibiting activity against COX. Therefore compound (IX) or salt thereof is also useful as medicament.

Compound (II) can be synthesized from compound (VI) and (VII) by following process other than purchase.

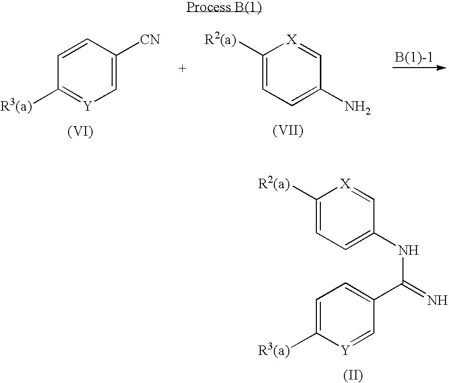

In the above formulae, R²(a), R³(a), X and Y represent the same meanings as defined above.

Process B(1) is the process for preparing the compound (II), which is the starting material of Process A(1) and A(2).

Compound (VI) and (VII) may be purchased if it is commercial, or synthesized according to general methods from commercial compounds, because the compounds as starting compound for synthesis of compound (II) have comparatively simple structure.

In this process, first, to the solution of compound (VII) is added strong base.

The strong base employable in this process is not particularly limited and may include alkali metal hydrides such as lithium hydride, sodium hydride; alkali metal alkoxides such as lithium methoxide, sodium methoxide, sodium ethoxide, potassium t-butoxide; or the like.

The solvent employable in this process is not particularly limited so long as it is inactive in this reaction and may include ethers such as diethyl ether, diisopropyl ether, tatrahydrofuran, dioxane; amides such as N,N-dimethylformamide, dimethylacetamide, hexamethylphosphoric triamide; sulfoxides such as dimethylsulfoxide; or the like.

The reaction temperature varies depending on the starting material, the solvent, etc., but it is usually from −10° C. to room temperature, preferably room temperature.

The reaction time varies depending on the starting material the solvent, the reaction temperature, etc., but it is usually from 5 min to 1 hr, preferably from 10 min to 40 min.

Preferably, this process is carried out under inert gas such as nitrogen gas.

In this process, then to the reaction mixture is added compound (VI).

The reaction temperature varies depending on the starting material, the solvent, etc., but it is usually from −10° C. to room temperature, preferably room temperature.

The reaction time varies depending on the starting material, the solvent, the reaction temperature, etc., but it is usually from 1 hr to 24 hrs, preferably from 2 hrs to overnight.

After the reaction, the reaction mixture is poured into ice water to decompose the excess strong base. Then, the desired compound may be collected by filtration as precipitate. Where necessary, it may be washed by solvent such as diisopropyl ether. Further, the desired compound is purified by the conventional method such as silica gel column chromatography, recrystallization, or the like, however, it may be used in the next step without further purification.

Compound (II) can be also synthesized from compound (VII) and (VIII) by following process other than purchase.

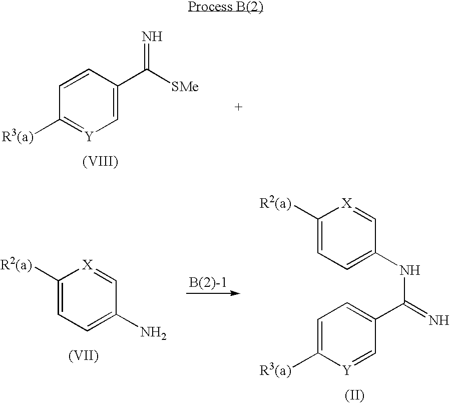

In the above formulae, R²(a), R³(a),X and Y represent the same meanings as defined above.

Process B(2) is the another process for preparing the compound (II) in the case that R²(a) is the group such as [(lower)alkoxy]carbonyl or the like, which tends to be nucleophilically attacked more easily than cyano group.

In this process, compound (VII) and (VIII) are condensated in the acidic condition.

Compound (VII) may be purchased if it is commercial, or synthesized according to general methods from commercial compounds.

Compound (VIII) may be synthesized by conventional method, that is, first the nitrile compound is led to thioamide compound by thioacetamide, and then methylated.

The solvent employable in this process is not particularly limited so long as it is inactive in this reaction and may include alcohols such as methanol, ethanol, 2-propanol; ethers such as diisopropyl ether, tatrahydrofuran, dioxane; and mixed solvent thereof; or the like.

The acid for making acidic condition in this process is not particularly limited so long as it is used in a usual reaction as an acid catalyst and may include inorganic acid such as hydrochloric acid, hydrobromic acid, sulfuric acid, or the like.

The reaction temperature varies depending on the starting material, the solvent, etc., but it is usually from 50° C. to 150° C., preferably reflux condition.

The reaction time varies depending on the starting material, the solvent, the reaction temperature, etc., but it is usually from 30 min to 5 hrs, preferably from 2 hrs to 4 hrs.

After the reaction, the reaction mixture is poured into basic water and extracted with organic solvent insoluble with water such as ethyl acetate. The organic layer is dried over anhydrous magnesium sulfate or anhydrous sodium sulfate, evaporated in vacuo. Where necessary, it may be washed by solvent such as diisopropyl ether. Further, the desired compound is purified by the conventional method such as silica gel column chromatography, recrystallization, etc, however, it may be used in the next step without further purification.

Above processes (Process A and B), all starting materials and product compounds may be salts. The compounds of above processes can be converted to salt according to a conventional method.

In the above compounds which have reactive group, may be protected at the group on cue and be deprotected on cue. In these reactions (protecting or deprotecting steps), concerning the kind of protective group and the condition of the reaction, ┌PROTECTIVE GROUPS IN ORGANIC SYNTHESIS Second Edition┘ T. W. Green and P. G. M. Wuts, John Wiley & Sons, INC. may be referred.

For therapeutic purpose, the compound (I) and a pharmaceutically acceptable salt thereof of the present invention can be used in a form of pharmaceutical composition containing one of said compounds as an active ingredient, in admixture with a pharmaceutically acceptable carrier such as an organic or inorganic solid or liquid excipient suitable for oral, parenteral or external administration. The pharmaceutical preparations may be capsules, tablets, dragees, granules, inhalant, suppositories, solution, lotion, suspension, emulsion, ointment, gel, cream, or the like. If desired, there may be included in these preparations, auxiliary substances, stabilizing agents, wetting or emulsifying agents, buffers and other commonly used additives.

Further the commercial package comprising the pharmaceutical composition mentioned above and a written matter, which states above mentioned effects, is also useful.

While the dosage of therapeutically effective amount of the compound (I) will vary depending upon the age and condition of each individual patient, or the like, an average single dose of about 0.01 mg, 0.1 mg, 1 mg, 10 mg, 50 mg, 100 mg, 250 mg, 500 mg and 1000 mg of the compound (I) may be effective for treating the above-mentioned diseases. In general, amounts between 0.01 mg/body and about 1,000 mg/body may be administered per day.

### THE BEST MODE FOR CARRYING OUT THE INVENTION

The following Examples are given only for the purpose of illustrating the present invention in more detail.

Although the present invention has been fully described by way of example, it is to be understood that various changes and modifications will be apparent to those skilled in the art. Therefore, unless otherwise such changes and modifications depart from the scope of the present invention hereinafter defined, they should be construed as being included therein.

### EXAMPLE 1-1

### N¹-(4-Bromophenyl)-4-methoxybenzamidine

Under Nitrogen gas, to a solution of 4-bromoaniline (3.88 g, 22.5 mmol) in dimethylsulfoxide (30 ml) was added NaH (568 mg, 23.7 mmol) at room temperature. After the mixture was stirred for 30 min, 4-methoxybenzonitrile (3.0 g, 22.5 mmol) was added.

The reaction mixture was stirred overnight then poured into 300 ml of ice-water. The precipitates were collected by filtration and washed with isopropyl ether to give 5.53 g of desired compound as a white solid (80.4%).

IR (KBr, cm⁻¹): 3473, 3357, 2958, 1612, 1249, 1174, 1103, 1074, 1030, 837.

NMR (DMSO-d₆, δ): 3.80(3H, s), 6.32(2H, brs), 6.78(2H, d, J=9 Hz), 6.96(2H, d, J=9 Hz), 7.42(1H, d, J=8 Hz), 7.92(2H, d, J=8 Hz).

MS: 305 (M+H)⁺ (⁷⁹Br), 307 (M+H)⁺ (⁸¹Br).

### EXAMPLE 1-2

### 1-(4-Bromophenyl)-2-(4-methoxyphenyl)-4-trifluoromethyl-1H-imidazole

To a mixture of N¹-(4-Bromophenyl)-4-methoxybenzamidine obtained by Example 1-1 (2.0 g, 6.55 mmol) and sodium bicarbonate (826 mg, 9.83 mmol) in 2-propanol (20 ml) was added 3-bromo-1,1,1-trifluoro-2-propanone (2.0 g, 10.5 mmol). The reaction mixture was heated at 80° C. for 2 hrs.

The reaction mixture was cooled to room temperature and filtered. The organic layer was evaporated in vacuo. The residue in acetic acid (20 ml) was heated at 110° C. for 2.5 hrs.

The reaction mixture was poured into ice-water (100 ml) and neutralized with sodium hydroxide aq. and extracted with ethyl acetate (50 ml). The organic layer was washed with brine, dried by magnesium sulfate and evaporated in vacuo. The residue was purified by silica gel column chromatography (20 g) eluting with n-hexane/ethyl acetate (10/1) and washed with diisopropyl ether to give 660 mg of desired compound (25.4%).

MP: 140-141° C.

IR (KBr, cm⁻¹): 3140, 2970, 1487, 1294, 1252, 1149, 1122, 1026, 833.

NMR (DMSO-d₆, δ): 3.75(3H, s), 6.92(2H, d, J=9 Hz), 7.27(2H, d, J=9 Hz), 7.36(2H, d, J=9 Hz), 7.71(2H, d, J=2 Hz), 8.18(1H, s).

MS: 397 (M+H)⁺ (⁷⁹Br), 399 (M+H)⁺ (⁸¹Br).

### EXAMPLE 2-1

### 4-Methoxy-N¹-(2-Methoxy-5-pyridinyl)benzamidine

Reaction was carried out in a manner similar to Example 1-1 using 4-methoxybenzonitrile and 5-amino-2-methoxypyridine to give 4.57 g of desired compound (78.8%).

IR (KBr, cm⁻¹): 3452, 3334, 3205, 2946, 1606, 1483, 1273, 1246, 1176, 1028, 841.

NMR (DMSO-d₆, δ): 3.80(3H, s), 3.82(3H, s), 6.36(2H, brs), 6.76(1H, d, J=9 Hz), 6.96(2H, d, J=9 Hz), 7.20(1H, dd, J=9 Hz and 3 Hz), 7.67(1H, d, J=3 Hz), 7.94(2H, d, J=9 Hz).

MS: 258 (M+H)⁺.

### EXAMPLE 2-2

### 2-(4-Methoxyphenyl)-1-(2-methoxy-5-pyridinyl)-4-trifluoromethyl-1H-imidazole hydrochloride

Reaction was carried out in a manner similar to Example 1-2 using 4-methoxy-N¹-(2-Methoxy-5-pyridinyl)benzamidine obtained by Example 2-1 to give 2-(4-methoxyphenyl)-1-(2-methoxy-5-pyridinyl)-4-trifluoromethyl-1H-imidazole.

Then, the product obtained was dissolved in ethyl acetate and treated with 4N hydrogen chloride in ethyl acetate to give 399 mg of desired compound as a white amorphous solid (14.7%).

NMR (DMSO-d₆, δ): 3.75(3H, s), 3.89(3H, s), 6.80-7.05(3H, m), 7.31(2H, d, J=9 Hz), 7.43(1H, d. J=9 Hz), 7.74(1H, dd, J=9 Hz and 2 Hz), 8.17(1H, s), 8.27(1H, s).

MS: 350 (M+H)⁺ (free).

### EXAMPLE 3-1

### N¹-(4-Methoxyphenyl)-2-methoxy-5-amidinopyridine

Under Nitrogen gas, to a solution of p-anisidine (2.75 g, 22.4 mmol) in tetrahydrofuran (15 ml) was added dropwise 1.0M sodium bis(trimethylsilyl)amide in tetrahydrofuran (23.5 ml, 23.5 mmol) at room temperature. After the mixture was stirred for 20 min, 6-methoxynicotinonitrile (3.0 g, 22.4 mmol) was added.

The reaction mixture was stirred for 4 hrs, then poured into 300 ml of ice-water. The precipitates were collected by filtration, washed with diisopropyl ether to give 3.36 g of desired compound (58.4%) (mixture).

This material was used without further purification.

NMR (DMSO-d₆, δ): 3.73(3H, s), 3.90(3H, s), 6.27(2H, brs), 6.70-7.00(5H, m), 8.24(1H, dd, J=9 Hz and 2 Hz), 8.72(1H, d, J=2 Hz).

MS: 258 (M+H)⁺.

### EXAMPLE 3-2

### 1-(4-Methoxyphenyl)-2-(2-methoxy-5-pyridinyl)-4-trifluoromethyl-1H-imidazole

Reaction was carried out in a manner similar to Example 1-2 using N¹-4-methoxy-2-methoxy-5-amidinopyridine obtained by Example 3-1 to give 526.6 mg of desired compound as a colorless crystal (21.5%).

MP: 90-92° C.

IR (KBr, cm⁻¹): 3141, 3107, 1604, 1518, 1294, 1248, 1159, 1118, 835.

NMR (DMSO-d₆, δ): 3.81(3H, s), 3.83(3H, s), 6.81(1H, d, J=9 Hz), 7.05(2H, d, J=9 Hz), 7.38(2H, d, J=9 Hz), 7.65(1H, dd, J=9 Hz and 2 Hz), 8.08(1H, d, J=2 Hz), 8.17(1H, s).

MS: 350 (M+H)⁺.

### EXAMPLE 4-1

### 4-Cyano-4,5-dihydro-1-(4-methoxyphenyl)-2-(2-methoxy-5-pyridinyl)-1H-imidazole

To a suspension of N¹-4-Methoxy-2-methoxy-5-amidinopyridine obtained by Example 3-1 in tetrahydrofuran (20 ml) were added 2-chloroacrylonitrile and diisopropylethylamine successively. The reaction mixture was heated at 70° C. After 5 hrs, an additional 1.07 ml of 2-chloroacrylonitrile was added and refluxed overnight.

The reaction mixture was cooled to room temperature, filtered and the solvent was removed in vacuo. The crude mixture was purified by silica gel column chromatography (24 g) eluting with ethyl acetate to give 460 mg of desired compound (54.9%).

This material was used in Example 4-2 without further purification.

### EXAMPLE 4-2

### 4-Cyano-1-(4-methoxyphenyl)-2-(2-methoxy-5-pyridinyl)-1H-imidazole

A suspension of the residue obtained by Example 4-1 and manganese(IV) oxide (MnO₂) (1.3 g, 10 eq) intoluene (10 ml) was heated at 85° C. for 5.5 hrs. To the reaction mixture was added manganese(IV) oxide (0.65 g, 5 eq) and heated at 110° C. for 3 hrs.

After cooling, the mixture was filtered through a Celite. The organic fraction was concentrated (396 mg). The crude mixture was purified by silica gel column chromatography (12 g) eluting with chloroform/methanol (50/1→15/1) and washed with diisopropyl ether to give 200.8 mg of desired compound as a colorless solid (24.1%, through Example 4-1 and 4-2).

MP: 130-132° C.

IR (KBr, cm⁻¹): 3132, 2949, 2233, 1604, 1516, 1466, 1292, 1254, 1024, 835.

NMR (DMSO-d₆, δ): 3.81(3H, s), 3.84(3H, s), 6.81(1H, d, J=9 Hz), 7.06(2H, d, J=9 Hz), 7.37(2H, d, J=9 Hz), 7.62(1H, dd, J=9 Hz and 2 Hz), 8.10(1H, d, J=2 Hz), 8.47(1H, s).

MS: 307 (M+H)⁺.

### EXAMPLE 5-1

### N¹-(4-Benzyloxyphenyl)-2-methoxy-5-amidinopyridine

Reaction was carried out in a manner similar to Example 3-1 using 4-benzyloxyaniline hydrochloride to give 8.7 g of desired compound (71.7%).

IR (KBr, cm⁻¹): 3488, 3396, 3031, 2958, 1635, 1502, 1373, 1236, 1103, 1020, 840.

NMR (DMSO-d₆, δ): 3.90(3H, s), 5.06(2H, s), 6.28(2H, brs), 6.70-7.05(5H, m), 7.25-7.60(5H, m), 8.24(1H, dd, J=9 Hz and 2 Hz), 8.72(1H, d, J=2 Hz).

MS: 334 (M+H)⁺.

### EXAMPLE 5-2

### 1-(4-Benzyloxyphenyl)-2-(2-methoxy-5-pyridinyl)-4-trifluoromethyl-1H-imidazole

Reaction was carried out in a manner similar to Example 1-2 using N¹-(4-benzyloxyphenyl)-2-methoxy-5-amidinopyridine obtained by Example 5-1 to give 2.27 g of desired compound (44.5%).

IR (KBr, cm⁻¹): 3064, 2950, 1290, 1244, 1157, 1122, 1022, 835.

NMR (DMSO-d₆, δ): 3.84(3H, s), 5.16(2H, s), 6.81(1H, d, J=9 Hz), 7.05-7.58(9H, m), 7.65(1H, dd, J=9 Hz and 2 Hz), 8.08(1H, d, J=2 Hz), 8.17(1H, s).

MS: 426 (M+H)⁺.

### EXAMPLE 6

### 1-(4-Hydroxyphenyl)-2-(2-methoxy-5-pyridinyl)-4-trifluoromethyl-1H-imidazole

To a solution of 1-(4-benzyloxyphenyl)-2-(2-methoxy-5-pyridinyl)-4-trifluoromethyl-1H-imidazole obtained by Example 5-2 (2.25 g, 5.29 mmol) in cyclohexene (22 ml) and ethanol (45 ml) was added 20% palladium hydroxide on carbon (550 mg). The resulting mixture was stirred at reflux for 2 hrs.

After cooling to room temperature, the mixture was filtered through Celite and washed with ethanol. The filtrate was concentrated in vacuo, and then the residue was washed with diisopropyl ether to give 1.31 g of desired compound as a white solid (73.9%).

MP: 198-200° C.

IR (KBr, cm⁻¹): 3600-2600, 1469, 1292, 1247, 1159, 1126, 833.

NMR ((CDCl₃, δ): 3.91(3H, s), 6.67(1H, brs), 6.73(1H, d, J=9 Hz), 6.87(2H, d, J=9 Hz), 7.11(2H, d, J=9 Hz), 7.43(1H, s), 7.86(1H, dd, J=9 Hz and 2 Hz), 8.03(1H, d, J=2 Hz).

MS: 336 (M+H)⁺.

### EXAMPLE 7

### 2-(2-Methoxy-5-pyridinyl)-1-(4-trifluoromethanesulfonyloxyphenyl)-4-trifluoromethyl-1H-imidazole

To the mixture of 1-(4-hydroxyphenyl)-2-(2-methoxy-5-pyridinyl)-4-trifluoromethyl-1H-imidazole obtained by Example 6 (600 mg, 1.79 mmol) and triethylamine (190 mg, 1.88 mmol) in chloroform (12 ml) was added trifluoromethanesulfonic anhydride dropwise at an ice bath temperature and stirred for 4.5 hrs.

Sodium hydrogencarbonate aq. (10 ml) was added to quench the reaction. The reaction mixture was partitioned between chloroform and water. The organic layer was washed with water and then brine, dried by magnesium sulfate and evaporated in vacuo. The residue was purified by silica gel column chromatography (10 g) eluting with n-hexane/ethyl acetate (10/1) to give 593 mg of desired compound (70.9%).

IR (KBr, cm⁻¹): 3118, 3062, 1421, 1255, 1219, 1136, 891.

NMR (CDCl₃, δ): 3.92(3H, s), 6.71(1H, d, J=9 Hz), 7.30-7.48(4H, m), 7.50(1H, s), 7.66(1H, dd, J=9 Hz and 2 Hz), 8.08(1H, d, J=2 Hz).

MS: 467 (M+H)⁺.

### EXAMPLE 8

### 1-(4-Cyanophenyl)-2-(2-methoxy-5-pyridinyl)-4-trifluoromethyl-1H-imidazole

To a solution of 2-(2-methoxy-5-pyridinyl)-1-(4-trifluoromethanesulfonyloxyphenyl)-4-trifluoromethyl-1H-imidazole obtained by Example 7 (150 mg, 0.321 mmol) in N,N-dimethylformamide (7.5 ml) were added zinc cyanide (Zn(CN)₂) (38 mg, 0.321 mmol) and tetrakis(triphenylphosphine)palladium (Pd(PPh₃)₄) (185 mg, 0.16 mmol) at room temperature under nitrogen gas. The mixture was stirred at 85° C. for 2 days.

The mixture was cooled to room temperature and partitioned between ethyl acetate (50 ml) and water (50 ml). The organic layer was washed with water and brine, then dried by magnesium sulfate, and evaporated in vacuo. The residue was purified by silica gel column chromatography (20 g) eluting with toluene/ethyl acetate (10:1) and washed with diisopropyl ether to give 57.2 mg of desired compound as a white solid (51.8%).

MP: 155-158° C.

IR (KBr, cm⁻¹): 3120, 2250, 1606, 1250, 1122, 822.

NMR (DMSO-d₆, δ): 3.85(3H, s), 6.82(1H, d, J=9 Hz), 7.61(1H, dd, J=9 Hz and 2 Hz), 7.65(2H, d, J=9 Hz), 8.03(2H, d, J=9 Hz), 8.12(1H, d, J=2 Hz), 8.36(1H, s).

MS: 345 (M+H)⁺.

### EXAMPLE 9

### 4-Ethoxycarbonyl-1-(4-methoxyphenyl)-2-(4-methoxyphenyl)-1H-imidazole

A mixture of N¹-(4-methoxyphenyl)-4-methoxybenzamidine (0.65 g), ethyl bromopyruvate (0.64 ml) and sodium hydrogencarbonate (0.85 g) in ethanol (7 ml) was stirred at reflux condition for overnight.

After cooling to room temperature, the reaction mixture was filtrated and evaporated in vacuo. Then the residue was poured into water, extracted with ethyl acetate, dried over magnesium sulfate, and evaporated in vacuo. The residue was purified by silica gel column chromatography eluting with n-hexane/ethyl acetate (5/1→2/1) to give 244 mg of desired compound as an oil (27.3%).

IR (Neat, cm⁻¹): 3437, 3392, 3367, 3217, 3140, 3072, 2966, 2843, 1803, 1699, 1651, 1614.

NMR (DMSO-d₆, δ): 1.29(3H t, J=7.1 Hz), 3.74(3H, s), 3.80(3H, s), 4.27(2H, q, J=7.1 Hz), 6.88(2H, dd, J=6.8 Hz and 2.1 Hz), 7.02(2H, dd, J=6.7 Hz and 2.1 Hz), 7.26 (2H, dd, J=5.0 Hz and 2.1 Hz), 7.28 (2H, dd, J=6.7 Hz and 2.1 Hz), 8.02 (1H, s).

MS: 353 (M+H)⁺.

### EXAMPLE 10

### 4-Carbamoyl-1-(4-methoxyphenyl)-2-(4-methoxyphenyl)-1H-imidazole

A mixture of 4-ethoxycarbonyl-1-(4-methoxyphenyl)-2-(4-methoxyphenyl)-1H-imidazole obtained by Example 9 (244 mg) and sodium methoxide (112 mg) in formamide (2 ml) was stirred at 100° C. for 2 hrs.

After cooling to room temperature, the reaction mixture was poured into water, extracted with ethyl acetate, dried over magnesium sulfate, and evaporated in vacuo. The residue was purified by silica gel column chromatography eluting with n-hexane/ethyl acetate (1/1→0/1) to give 73 mg of desired compound (32.6%).

MP: 167-169° C.

IR (KBr, cm⁻¹): 3427, 3342, 3276, 3155, 2964, 2841, 1672, 1610.

NMR (DMSO-d₆, δ): 3.74(3H, s), 3.80(3H, s), 6.87-6.89(2H, m), 7.00-7.03(2H, m), 7.20(1H, s), 7.26-7.29(4H, m), 7.43(1H, s), 7.77(1H, s).

MS: 324 (M+H)⁺.

### EXAMPLE 11

### 4-Cyano-1-(4-methoxyphenyl)-2-(4-methoxyphenyl)-1H-imidazole hydrochloride

A mixture of 4-carbamoyl-1-(4-methoxyphenyl)-2-(4-methoxyphenyl)-1H-imidazole obtained by Example 10 (73 mg) and phosphorus oxychloride (63 μl) in N,N-dimethylformamide (1 ml) was stirred at room temperature for 1 hr.

The reaction mixture was poured into saturated aqueous sodium hydrogencarbonate extracted with ethyl acetate, dried over magnesium sulfate, and evaporated in vacuo. The residue was purified by silica gel column chromatography eluting with n-hexane/ethyl acetate (2/1).

After correcting the fraction, the solvent was removed by evaporation and the residue was dissolved in ethyl acetate (1 ml). 4N Hydrochloride/ethyl acetate (56 ml) was added to the above solution. Resulting precipitates were corrected by filtration and washed with isopropyl ether to give 38 mg of desired compound (49.2%).

MP: 142-143° C.

IR (KBr, cm⁻¹): 3425, 3407, 3132, 3076, 3043, 3026, 2962, 2929, 2835, 2231, 1608.

NMR (DMSO-d₆, δ): 3.74(3H, s), 3.80(3H, s), 6.55(1H, s), 6.88-6.91(2H, m), 7.03-7.05(2H, m), 7.25-7.32(4H, m), 8.39(1H, s).

MS: 306 (free) (M+H)⁺.

### EXAMPLE 12-1

### 4-Cyano-4,5-dihydro-1-(4-methoxyphenyl)-2-(4-methoxyphenyl)-1H-imidazole

A mixture of N¹-(4-methoxyphenyl)-4-methoxybenzamidine (5 g), 2-chlorocyanoethylene (2.0 ml) and N,N-diisopropylethylamine (4.38 ml) in tetrahydrofuran (100 ml) was stirred at reflux condition for 6 hrs. Additional 2-chlorocyanoethylene (2.01 ml) was added, the mixture was refluxed for overnight.

After cooling to room temperature, the reaction mixture was evaporated in vacuo. The residue was purified by silica gel column chromatography eluting with n-hexane/ethyl acetate (1/1) to give 3.28 g of desired compound as an oil (63.7%).

IR (Neat, cm⁻¹): 3283, 3217, 3114, 3055, 3003, 2958, 2839, 2243, 2048, 1896, 1732, 1606.

NMR (DMSO-d₆, δ): 3.70(3H, s), 3.74-(3H, s), 4.11-4.19(2H, m), 5.20(1H, dd, J=10.5 Hz and 8.2 Hz), 6.81-6.97(6H, m), 7.32-7.37(2H, m).

MS: 308 (M+H)⁺.

### EXAMPLE 12-2

### 4-Cyano-1-(4-methoxyphenyl)-2-(4-methoxyphenyl)-1H-imidazole

A suspension of 4-cyano-4,5-dihydro-1-(4-methoxyphenyl)-2-(4-methoxyphenyl)-1H-imidazole obtained by Example 12-1 (2.7 g) and manganese(IV) oxide (MnO₂) (3.82 g) in N,N-dimethylformamide (30 ml) was stirred at 100° C. for 4 hrs.

After filtration, the reaction mixture was poured into water, extracted with ethyl acetate, dried over magnesium sulfate, and evaporated in vacuo. To the solution of the residue in N,N-dimethylformamide (30 ml), phosphorus oxychloride (2.46 ml) was added under stirring at 0° C.

After stirring at room temperature for 1 hr, the reaction mixture was poured into saturated aqueous sodium hydrogencarbonate, extracted with ethyl acetate, dried over magnesium sulfate, and evaporated in vacuo to give 2.11 g of desired compound (78.7%).

MP: 132-134° C.

NMR (DMSO-d₆, δ): 3.74(3H, s), 3.80(3H, s), 6.87-6.93(2H, m), 7.02-7.08(2H, m), 7.23-7.34(4H, m), 8.39(1H, s).

MS: 306 (M+H)⁺.

### EXAMPLE 13

### 4-Cyano-1-(4-methoxyphenyl)-2-(4-methoxyphenyl)-1H-imidazole hydrochloride

4N Hydrochloride/ethyl acetate (254 μl) was added to a solution of 4-cyano-1-(4-methoxyphenyl)-2-(4-methoxyphenyl)-1H-imidazole obtained by Example 12-2 (300 mg) in ethyl acetate (1 ml). Resulting precipitates were corrected by filtration and washed with isopropyl ether to give 300 mg of desired compound (86.4%).

MP: 142-143° C.

IR (KBr, cm⁻¹): 3425, 3407, 3132, 3076, 3043, 3026, 2962, 2929, 2835, 2231, 1608.

NMR (DMSO-d₆, δ): 3.74(3H, s), 3.80(3H, s), 6.55(1H, s), 6.88-6.91(2H, m), 7.03-7.05(2H, m), 7.25-7.32(4H, m), 8.39(1H, s).

MS: 306 (free) (M+H)⁺.

### EXAMPLE 14

### 4-Ethoxycarbonyl-1-(4-methoxyphenyl)-2-(4-methoxyphenyl)-1H-imidazole

A mixture of 4-cyano-1-(4-methoxyphenyl)-2-(4-methoxyphenyl)-1H-imidazole (315 mg) and 4N hydrochloride/ethanol (6.2 ml) was stirred at reflux condition for 1 hr.

After cooling to room temperature, the reaction mixture was poured into saturated aqueous sodium hydrogencarbonate, extracted with ethyl acetate, dried over magnesium sulfate, and evaporated in vacuo. The residue was purified by silica gel column chromatography eluting with n-hexane/ethyl acetate (1/1) to give 0.26 g of desired compound (71.5%).

MP: 142-143° C.

IR (Neat, cm⁻¹): 3437, 3392, 3367, 3217, 3140, 3072, 2966, 2843, 1803, 1699, 1651, 1614.

NMR (DMSO-d₆, δ): 1.29(3H, t, J=7.1 Hz), 3.74(3H, s), 3.80(3H, s), 4.27(2H, q, J=7.1 Hz), 6.88(2H, dd, J=6.8 Hz and 2.1 Hz), 7.02(2H, dd, J=6.7 Hz and 2.1 Hz), 7.26(2H, dd, J=5.0 Hz and 2.1 Hz), 7.28(2H, dd, J=6.7 Hz and 2.1 Hz), 8.02(1H, s).

MS: 353 (M+H)⁺.

### EXAMPLE 15

### 4-Hydroxymethyl-1-(4-methoxyphenyl)-2-(4-methoxyphenyl)-1H-imidazole

1N Diisopropylalminiumhydride in toluene (3.76 ml) was added dropwise to a solution of 4-ethoxycarbonyl-1-(4-methoxyphenyl)-2-(4-methoxyphenyl)-1H-imidazole obtained by Example 14 (5 ml) under stirring at −78° C., and stirred at −78° C. for 2 hrs.

The reaction mixture was quenched by saturated aqueous ammonium chloride, then 1N hydrochloric acid was added and extracted with water. The combined aqueous layer was neutralized with saturated aqueous sodium hydrogencarbonate, extracted with ethyl acetate, and dried over magnesium sulfate. After evaporation of the solution, the residue was purified by silica gel column chromatography eluting with n-hexane/ethyl acetate (1/1) to give 0.14 g of desired compound (30%).

IR (Neat, cm⁻¹): 3369, 3307, 3224, 3076, 3006, 2939, 2837, 1676, 1608.

NMR (DMSO-d₆, δ): 3.73(3H, s), 3.79(3H, s), 4.42(2H, d, J=5.6 Hz), 4.96(1H, t, J=5.6 Hz), 6.85(2H, d, J=8.8 Hz), 7.00(2H, d, J=8.9 Hz), 7.15-7.25(5H, m).

MS: 311 (M+H)⁺.

### EXAMPLE 16

### 4-Formyl-1-(4-methoxyphenyl)-2-(4-methoxyphenyl)-1H-imidazole

Dimethylsulfoxide (125 μl) was added to a solution of oxalylchloride (118 μl) in dichloromethane (2 ml) under stirring at −78° C. After stirred at −78° C. for 10 min, a solution of 4-hydroxymethyl-1-(4-methoxyphenyl)-2-(4-methoxyphenyl)-1H-imidazole obtained by Example 15 (0.21 g) in dichloromethane (2 ml) was added and stirred at −78° C. for 1 hr. Triethylamine (0.66 ml) was added to the reaction mixture, and stirred at 0° C. for 20 min.

The mixture was quenched by saturated aqueous ammonium chloride, extracted with ethyl acetate, dried over magnesium sulfate, and evaporated in vacuo to give 120 mg of desired compound as an oil (57.5%).

IR (Neat, cm⁻¹): 3126, 3057, 3005, 2960, 2837, 2760, 2551, 2048, 1685, 1610.

NMR (CDCl₃, δ): 3.83(3H, s), 3.86(3H, s), 6.81(2H, dd, J=6.9 Hz and 2.0 Hz), 6.94(2H, dd, J=6.8 Hz and 2.1 Hz), 7.16(2H, dd, J=6.7 Hz and 2.2 Hz), 7.36(2H, dd, J=6.7 Hz and 2.1 Hz), 7.16(1H, s), 9.98(1H, s).

MS: 309 (M+H)⁺.

### EXAMPLE 17

### 4-Difluoromethyl-1-(4-methoxyphenyl)-2-(4-methoxyphenyl)-1H-imidazole hydrochloride

Diethylaminosulfur trifluoride (154 μl) was added to a solution of 4-formyl-1-(4-methoxyphenyl)-2-(4-methoxyphenyl)-1H-imidazole obtained by Example 16 (120 mg) in dicloromethane (2 ml) under stirring at 0° C.

After stirring at room temperature for overnight, the reaction mixture was poured into saturated aqueous sodium hydrogencarbonate, extracted with ethyl acetate, dried over magnesium sulfate, and evaporated in vacuo. The residue was purified by silica gel column chromatography eluting with n-hexane/ethyl acetate (1/1). After correcting the fraction, the solvent was removed by evaporation and the residue was dissolved in ethyl acetate (1 ml). 4N hydrochloride/ethyl acetate (97 μl) was added. Resulting precipitates were corrected by filtration and washed with isopropyl ether to give 24 mg of desired compound (16.8%).

MP: 150-153° C.

IR (KBr, cm⁻¹): 3454, 3433, 3265, 3101, 3060, 2958, 2837, 2735, 2659, 2563, 1606.

NMR (DMSO-d₆, δ): 3.76(3H, s), 3.80(3H, s), 6.84(1H, t, J=56.2 Hz), 6.91-6.97(2H, s), 7.02-7.08(2H, m), 7.28-7.38(4H, m), 7.93 (1H, t, J=2.2 Hz).

MS: 331 (free) (M+H)⁺.

### EXAMPLE 18

### 4-Carboxy-1-(4-methoxyphenyl)-2-(4-methoxyphenyl)-1H-imidazole

A mixture of 4-cyano-1-(4-methoxyphenyl)-2-(4-methoxyphenyl)-1H-imidazole (1.5 g) in 50% sulfuric acid (16 ml) was stirred at reflux condition for 1 hr.

After cooling to room temperature, the reaction mixture was poured into 6% sodium hydroxide aqueous solution (100 ml), and washed with ethyl acetate. The aqueous layer was acidified by concentrated hydrochloric acid, extracted with ethyl acetate, dried over magnesium sulfate, and evaporated in vacuo. The resulting precipitates were corrected by filtration and washed with isopropyl ether to give 1.18 g of desired compound (74.1%).

MP: 102-105° C.

IR (KBr, cm⁻¹): 3427, 3269, 3174, 3141, 3086, 3005, 2965, 2910, 2839, 1678, 1610.

NMR (DMSO-d₆, δ): 3.76(3H, s), 3.813(3H, s), 6.89(2H, dt, J=7.0 Hz and 2.0 Hz), 7.03(2H, dt, J=7.2 Hz and 2.0 Hz), 7.26-7.32(4H, m), 7.97(1H, s).

MS: 325 (M+H)⁺.

### EXAMPLE 19

### 4-Ethylmethycarbamoyl-1-(4-methoxyphenyl)-2-(4-methoxyphenyl)-1H-imidazole

A mixture of 4-carboxy-1-(4-methoxyphenyl)-2-(4-methoxyphenyl)-1H-imidazole obtained by Example 18 (170 mg), N-ethylmethylamine (45 μl), 1-hydroxybenzotriazole (71 mg) and 1-(3-dimethylaminopropyl)-3-ethylcarbodiimide hydrochloride (100 mg) in N,N-dimethylformamide (5 ml) was stirred at room temperature for overnight.

The reaction mixture was poured into water, extracted with ethyl acetate, dried over magnesium sulfate, and evaporated in vacuo. The residue was purified by silica gel column chromatography eluting with n-hexane/ethyl acetate (1/1). The resulting precipitates were corrected by filtration and washed with isopropyl ether to give 72 mg of desired compound (37.6%).

MP: 138-139° C.

IR (KBr, cm⁻¹): 3124, 3068, 3006, 2966, 2929, 2841, 1603.

NMR (DMSO-d₆, δ): 1.05-1.29(3H, m), 2.91-3.03(2H, m), 3.33-3.56(2H, m), 3.74(3H, s), 3.80(3H, s), 3.91-4.06(1H, m), 6.88(2H, dt, J=8.8 Hz and 1.8 Hz), 7.02(2H, dt, J=8.8 Hz and 2.0 Hz), 7.23-7.30(4H, m), 7.72(1H, s).

MS: 366 (M+H)⁺.

### EXAMPLE 20

### 4-Cyclopropyl-1-(4-methoxyphenyl)-2-(4-methoxyphenyl)-1H-imidazole hydrochloride

A mixture of N¹-(4-methoxyphenyl)-4-methoxybenzamidine (1 g), 2-bromo-1-cyclopropylethanone (1.27 g) and sodium hydrogencarbonate (656 mg) in 2-propanol (10 ml) was stirred at reflux condition for overnight.

After cooling to room temperature, the reaction mixture was filtered off and evaporated in vacuo. Then the residue was poured into water, extracted with ethyl acetate, dried over magnesium sulfate, and evaporated in vacuo. The residue was dissolved in acetic acid (10 ml), and refluxed for 1 hr.

After cooling to room temperature, the mixture was poured into saturated aqueous sodium hydrogencarbonate, extracted with ethyl acetate, dried over magnesium sulfate, and evaporated in vacuo. The residue was purified by silica gel column chromatography eluting with n-hexane/ethyl acetate (3/1). After correcting the fraction the solvent was removed by evaporation and the residue was dissolved in ethyl acetate (5 ml). 4N hydrochloride/ethyl acetate (175 μl) was added. Resulting precipitates were corrected by filtration and washed with isopropyl ether to give 200 mg of desired compound (14.4%).

MP: 180-181° C.

IR (KBr, cm⁻¹): 3273, 3051, 2966, 2935, 2906, 2835, 2740, 2640, 2592, 1610.

NMR (DMSO-d₆, δ): 0.88-0.96(2H, m), 1.00-1.07(2H, m), 2.02-2.11(2H, m), 3.79(3H, s), 3.80(3H, s), 7.00-7.11(4H, m), 7.35-7.41(4H, m), 7.67(1H, s).

MS: 321 (free) (M+H)⁺.

### EXAMPLE 21

### 1-(4-Methoxyphenyl)-2-(4-methoxyphenyl)-4-methyl-1H-imidazole hydrochloride

85 mg of desired compound was obtained from N¹-(4-methoxyphenyl)-4-methoxybenzamidine (200 mg) and 1-bromoacetone (204 μl) in a manner similar to that of Example 20.

MP: 203-205° C.

IR (KBr, cm⁻¹): 3400, 3114, 3055, 2966, 2929, 2833, 2804, 2711, 2650, 2578, 2426, 1612.

NMR (DMSO-d₆, δ): 2.39(3H, s), 3.79(3H, s), 3.81(3H, s), 7.02-7.12(4H, m), 7.36-7.66(4H, m), 7.66(1H, s), 14.6-15.5(1H, br).

MS: 295 (free) (M+H)⁺.

### EXAMPLE 22-1

### N¹-(4-Ethoxycarbonylphenyl)-4-methoxybenzamidine

A mixture of methyl 4-methoxybenzenecarbimidothioate hydroiodide (3.9 g), ethyl 4-aminobenzoate (2.08 g) and acetic acid (2 ml) in 2-propanol (40 ml) was stirred at reflux condition for 2 hrs.

After cooling to room temperature, the reaction mixture was poured into saturated aqueous sodium hydrogencarbonate, extracted with ethyl acetate, dried over magnesium sulfate, and evaporated in vacuo. Resulting precipitates were corrected by filtration and washed with isopropyl ether to give 2.35 g of desired compound (62.4%).

MP: 128-132° C.

IR (KBr, cm⁻¹): 3456, 3305, 3251, 3178, 2976, 2933, 2850, 1711, 1626.

NMR (DMSO-d₆, δ): 1.31(3H, t, J=7.1 Hz), 3.81(3H, s), 4.28(2H, q, J=7.1 Hz), 6.46(2H, s), 6.90-7.01(4H, m), 7.86-7.91(4H, m).

MS: 299 (M+H)⁺.

### EXAMPLE 22-2

### 1-(4-Ethoxycarbonylphenyl)-2-(4-methoxyphenyl)-4-trifluoromethyl-1H-imidazole

A mixture of N¹-(4-Ethoxycarbonylphenyl)-4-methoxybenzamidine obtained by Example 22-1 (0.5 g), 3-bromo-1,1,1-trifluoro-2-propanone (0.35 ml) and sodium hydrogencarbonate (563 mg) in 2-propanol (5 ml) was stirred at reflux condition for 4 hrs.

After cooling to room temperature, the reaction mixture was filtered off and evaporated in vacuo. Then the residue was poured into water, extracted with ethyl acetate, dried over magnesium sulfate, and evaporated in vacuo. The residue was dissolved in acetic acid (10 ml), and refluxed for 1 hr.

After cooling to room temperature, the mixture was poured into saturated aqueous sodium hydrogencarbonate, extracted with ethyl acetate, dried over magnesium sulfate, and evaporated in vacuo. The residue was purified by silica gel column chromatography eluting with n-hexane/ethyl acetate (3/1) to give 0.53 g of desired compound as an oil (81%).

IR (Neat, cm⁻¹): 3745, 3610, 3435, 3396, 3365, 3298, 3280, 3236, 3130, 2962, 2927, 2856, 1693, 1649.

NMR (DMSO-d₆, δ): 1.33(3H, t, J=7.1 Hz), 3.75(3H, s), 4.34(2H, q, J=7.1 Hz), 6.91(2H, dd, J=6.9 Hz and 1.9 Hz), 7.26(2H, dd, J=6.8 Hz and 2.0 Hz), 7.53 (2H, dd, J=6.8 Hz and 1.7 Hz), 8.04 (2H, dd, J=6.7 Hz and 1.8 Hz), 8.25 (1H, d, J=1.2 Hz).

MS: 391 (M+H)⁺.

### EXAMPLE 23

### 1-(4-Carbamoylphenyl)-2-(4-methoxyphenyl)-4-trifluoromethyl-1H-imidazole

255 mg of desired compound was obtained from 1-(4-ethoxycarbonylphenyl)-2-(4-methoxyphenyl)-4-trifluoromethyl-1H-imidazole obtained by Example 22-2 (710 mg) in a manner similar to that of Example 10.

IR (KBr, cm⁻¹): 3410, 3303, 3190, 3122, 2960, 2841, 1655, 1614.

NMR (DMSO-d₆, δ): 3.77(3H, s), 6.90(2H, dt, J=8.8 Hz and 2.0 Hz), 7.26(2H, dt, J=8.8 Hz and 2.1 Hz), 7.46(2H, d, J=8.5 Hz), 7.52(1H, s), 7.96(2H, d, J=8.5 Hz), 8.10(1H, s), 8.21(1H, d, J=1.2 Hz).

MS: 362 (M+H)⁺.

### EXAMPLE 24

### 1-(4-Cyanophenyl)-2-(4-methoxyphenyl)-4-trifluoromethyl-1H-imidazole

A mixture of 1-(4-carbamoylphenyl)-2-(4-methoxyphenyl)-4-trifluoromethyl-1H-imidazole obtained by Example 23 (200 mg) and phosphorus oxychloride (0.16 ml) in N,N-dimethylformamide (2 ml) was stirred at room temperature for 1 hr.

The reaction mixture was poured into saturated aqueous sodium hydrogencarbonate, extracted with ethyl acetate, dried over magnesium sulfate, and evaporated in vacuo. Resulting precipitates were corrected by filtration and washed with isopropyl ether to give 171 mg of desired compound (90%).

MP: 146-148° C.

IR (KBr, cm⁻¹): 3415, 3163, 3118, 3064, 3012, 2968, 2906, 2839, 2229, 1608.

NMR (DMSO-d₆, δ): 3.76(3H, s), 6.92(2H, dt, J=8.9 Hz and 1.9 Hz), 7.25(2H, dt, J=8.7 Hz and 2.0 Hz) , 7.60(2H, dt, J=8.5 Hz and 1.8 Hz) , 8.00(2H, dt, J=8.6 Hz and 1.7 Hz), 8.27(1H, d, J=1.1 Hz).

MS: 344 (M+H)⁺.

### EXAMPLE 25-1

### 4-Cyano-4 ,5-dihydro-1-(4-ethoxycarbonylphenyl)-2-(4-methoxyphenyl)-1H-imidazole

265 mg of desired compound was obtained from N¹-(4-ethoxycarbonylphenyl)-4-methoxybenzamidine (500 mg) in a manner similar to that of Example 12-1.

IR (Neat, cm⁻¹): 3417, 3253, 3217, 3068, 2974, 2902, 2841, 1711, 1603.

NMR (DMSO-d₆, δ): 1.28(3H, t, J=7.1 Hz), 3.78(3H, s), 4.26(2H, q, J=7.1 Hz), 4.31-4.46(2H, m) , 5.27(1H, t, J=9.9 Hz), 6.88-6.97(4H, m), 7.37(2H, dt, J=8.8 Hz and 1.9 Hz), 7.79 (2H, dt, J=8.7 Hz and 1.9 Hz).

MS: 350 (M+H)⁺.

### EXAMPLE 25-2

### 4-Cyano-1-(4-ethoxycarbonylphenyl)-2-(4-methoxyphenyl)-1H-imidazole

A suspension of 4-cyano-4,5-dihydro-1-(4-ethoxycarbonylphenyl)-2-(4-methoxyphenyl)-1H-imidazole obtained by Example 25-1 (0.26 g) and manganese(IV) oxide (MnO₂) (259 mg) in ethyl acetate (5 ml) was stirred at reflux condition for overnight.

After filtration, the reaction mixture was poured into water, extracted with ethyl acetate, dried over magnesium sulfate, and evaporated in vacuo. The residue was purified by silica gel column chromatography eluting with n-hexane/ethyl acetate (5/1) to give 117 mg of desired compound (45.3%).

MP: 139-140° C.

IR (KBr, cm⁻¹): 3425, 3143, 3060, 2979, 2947, 2902, 2839, 2235, 1718, 1606.

NMR (DMSO-d₆, δ): 1.33(3H, t, J=7.1 Hz), 3.75(3H, s), 4.34(2H, q, J=7.1 Hz), 6.90(2H, dt, J=8.8 Hz and 1.9 Hz), 7.25(2H, dt, J=8.8 Hz and 1.9 Hz), 7.52(2H, dt, J=8.5 Hz and 1.7 Hz), 8.05(2H, dt, J=8.5 Hz and 1.7 Hz), 8.55(1H, s).

MS: 348 (M+H)³⁰ .

### EXAMPLE 26

### 1-(4-Carbamoylphenyl)-4-cyano-2-(4-methoxyphenyl)-1H-imidazole

49 mg of desired compound was obtained from 4-cyano-1-(4-ethoxycarbonylphenyl)-2-(4-methoxyphenyl)-1H-imidazole obtained by Example 25-2 (100 mg) in a manner similar to that of Example 10 (53.5%).

MP: 228-290° C.

IR (KBr, cm⁻¹): 3456, 3396, 3354, 3292, 3172, 3113, 3051, 2970, 2837, 2227, 1682, 1612.

NMR (DMSO-d₆, δ): 3.75(3H, s), 6.91(2H, d, J=8.8 Hz), 7.26(2H, d, J=8.8 Hz), 7.46(2H, d, J=8.5 Hz), 7.54(1H, s), 7.97(2H, d, J=8.5 Hz), 8.11(1H, s), 8.52(1H, s).

MS: 319 (M+H)⁺.

### EXAMPLE 27

### 4-Cyano-1-(4-cyanophenyl)-2-(4-methoxyphenyl)-1H-imidazole

24 mg of desired compound was obtained from 1-(4-carbamoylphenyl)-4-cyano-2-(4-methoxyphenyl)-1H-imidazole obtained by Example 26 (40 mg) in a manner similar to that of Example 24 (63.6%).

MP: 185-186° C.

IR (KBr, cm⁻¹): 3419, 3219, 3132, 3091, 3057, 3012, 2968, 2935, 2837, 2229, 1608.

NMR (DMSO-d₆, δ): 3.76(3H, s), 6.92(2H, d, J=8.8 Hz), 7.25(2H, d, J=8.7 Hz), 7.59(2H, d, J=8.5 Hz), 8.02(2H, d, J=8.5 Hz), 8.56(1H, s).

MS: 301 (M+H)⁺.

### EXAMPLE 28

### 4-Acetyl-1-(4-methoxyphenyl)-2-(4-methoxyphenyl)-1H-imidazole

3N solution of methylmagunesium bromide in diethyl ether (1.17 ml) was added to a solution of 4-cyano-1-(4-methoxyphenyl)-2-(4-methoxyphenyl)-1H-imidazole (357 mg) in tetrahydrofuran (5 ml).

After stirring at room temperature for 2 hrs, the reaction mixture was poured into hydrochloric acid, extracted with ethyl acetate, washed with water, dried over magnesium sulfate, and evaporated in vacuo. The residue was purified by silica gel column chromatography eluting with n-hexane/ethyl acetate (5/1) to give 258 mg of desired compound (68.5%).

MP: 116-117° C.

IR (KBr, cm⁻¹): 3431, 3118, 3066, 3008, 2964, 2929, 2837, 1668, 1610.

NMR (DMSO-d₆, δ): 2.48(3H, s), 3.74(3H, s), 3.80(3H, s), 6.89(2H, d, J=8.6 Hz), 7.03(2H, d, J=8.8 Hz), 7.26-7.31(4H, m), 8.12(1H, s).

MS: 323 (M+H)⁺.

### EXAMPLE 29-1

### 4-Ethoxycarbonyl-4,5-dihydro-1-(4-benzyloxyphenyl)-2-(4-methoxyphenyl)-1H-imidazole

A mixture of N¹-(4-benzyloxyphenyl)-4-methoxybenzamidine (1.25 g), ethyl 2-chloroacrylate (0.76 g) and N,N-diisopropylethylamine (0.98 ml) in tetrahydrofuran (12 ml) was stirred at reflux condition for 2 hrs.

After cooling to room temperature, the reaction mixture was filtered off, the filtrate was poured into water, extracted with ethyl acetate, dried over magnesium sulfate, and evaporated in vacuo.

This material was used in the next step without further purification.

### EXAMPLE 29-2

### 4-Ethoxycarbonyl-1-(4-benzyloxyphenyl)-2-(4-methoxyphenyl)-1H-imidazole

The residue of Example 29-1 was dissolved in N,N-dimethylformamide (10 ml), and manganese(IV) oxide (1.63 g) was added to the solution.

After stirring at 100° C. for 4 hrs, the reaction mixture was cooled to room temperature and poured into water and ethyl acetate. After filtration, the mixture was extracted with ethyl acetate, dried over magnesium sulfate, and evaporated in vacuo. The residue was purified by silica gel column chromatography eluting with n-hexane/ethyl acetate (1/1) to give 1.5 g of desired compound as an oil (93.1%).

IR (Neat, cm⁻¹): 3433, 3253, 3224, 3140, 3064, 2966, 2843, 1722, 1712, 1606.

NMR (DMSO-d₆, δ): 1.29(3H, t, J=7.1 Hz), 3.75(3H, s), 4.27(2H, d, J=7.1 Hz), 5.15(2H, s), 6.88(2H, dt, J=8.9 Hz and 1.9 Hz), 7.10(2H, dt, J=8.9 Hz and 1.9 Hz), 7.24-7.49(9H, m), 8.04(1H, s).

MS: 429 (M+H)⁺.

### EXAMPLE 30

### 1-(4-Benzyloxyphenyl)-4-formyl-2-(4-methoxyphenyl)-1H-imidazole

0.95N Diisopropylalminiumhydride in toluene (6.49 ml) was added dropwise to a solution of 4-ethoxycarbonyl-1-(4-benzyloxyphenyl)-2-(4-methoxyphenyl)-1H-imidazole obtained by Example 29-2 (0.88 g) in dichloromethane (5 ml) under stirring at −78° C., and stirred at −78° C. for 2 hrs.

The reaction mixture was quenched by saturate aqueous ammonium chloride, then 1N hydrochloric acid was added, and extracted with water. After aqueous sodium hydroxide was added, extracted with ethyl acetate, dried over magnesium sulfate, and evaporated in vacuo.

The residue was dissolved in N,N-dimethylformamide (10 ml), and manganese(IV) oxide (1.79 g) was added to the solution.

After stirring at 100° C. for 1 hr, the reaction mixture was cooled to room temperature and poured into water and ethyl acetate. After filtration, the mixture was extracted with ethyl acetate, dried over magnesium sulfate, and evaporated in vacuo. The residue was purified by silica gel column chromatography eluting with n-hexane/ethyl acetate (1/1) to give 0.77 g of desired compound as an oil (97.5%).

IR (Neat, cm⁻¹): 3440, 3361, 3219, 3124, 3062, 2937, 2837, 2760, 1732, 1684, 1610.

NMR (DMSO-d₆, δ): 3.75(3H, s), 5.16(2H, s), 6.89(2H, dt, J=8.9 Hz and 1.9 Hz), 7.12(2H, dt, J=8.9 Hz and 2.1 Hz), 7.27-7.49(9H, m), 8.28(1H, s), 9.82(1H, s).

MS: 385 (M+H)⁺.

### EXAMPLE 31

### 1-(4-Benzyloxyphenyl)-4-difluoromethyl-2-(4-methoxyphenyl)-1H-imidazole

Diethylaminosulfur trifluoride (0.46 ml) was added to a solution of 1-(4-benzyloxyphenyl)-4-formyl-2-(4-methoxyphenyl)-1H-imidazole obtained by Example 30 (0.45 g) in dichloromethane (5 ml) under stirring at 0° C.

After stirring at room temperature for overnight, the reaction mixture was poured into saturated aqueous sodium hydrogencarbonate, extracted with ethyl acetate, dried over magnesium sulfate, and evaporated in vacuo. The residue was purified by silica gel column chromatography eluting with n-hexane/ethyl acetate (1/1) to give 0.38 g of desired compound as an oil (79.9%).

IR (Neat, cm⁻¹): 3433, 3155, 3113, 3066, 3041, 2964, 2841, 1732, 1610.

NMR (DMSO-d₆, δ): 3.74(3H, s), 5.15(2H, s), 6.87(2H, d, J=8.9 Hz), 7.08(1H, t, J=55.0 Hz), 7.10(2H, d, J=8.9 Hz), 7.24-7.45(9H, m), 7.73(1H, t, J=2.3 Hz).

MS: 407 (M+H)⁺.

### EXAMPLE 32

### 4-Difluoromethyl-1-(4-hydroxyphenyl)-2-(4-methoxyphenyl)-1H-imidazole

A suspension of 1-(4-benzyloxyphenyl)-4-difluoromethyl-2-(4-methoxyphenyl)-1H-imidazole obtained by Example 31 (0.38 g), dry 20% palladium hydroxide on carbon (Pd(OH)₂/C) (100 mg) in ethanol (8 ml) and cyclohexene (4 ml) was stirred at ref lux condition for 1 hr and cooled to room temperature.

After filtration, the reaction mixture was evaporated in vacuo to give 0.3 g of desired compound (ca.100%).

MP: 143-145° C.

IR (KBr, cm⁻¹): 3149, 3111, 3003, 2966, 2837, 2804, 2679, 2602, 1610.

NMR (DMSO-d₆, δ): 3.74(3H, s) , 6.80-6.91(4H, m), 6.96(1H, t, J=55.0 Hz) , 7.14(2H, dt, J=8.7 Hz and 1.9 Hz), 7.27(2H, dt, J=8.9 Hz and 1.9 Hz), 7.68(1H, t, J=2.2 Hz), 9.90(1H, s).

MS: 317 (M+H)⁺.

### EXAMPLE 33

### 4-Difluoromethyl-2-(4-methoxyphenyl)-1-(4-trifluoromethanesulfonyloxyphenyl)-1H-imidazole

Triethylamine (0.15 ml) and trifluoromethanesulfonic anhydride (0.18 ml) was added to a solution of 4-difluoromethyl-1-(4-hydroxyphenyl)-2-(4-methoxyphenyl)-1H-imidazole obtained by Example 32 (300 mg) in chloroform (5 ml) under stirring at 0° C.

After stirring at 0° C. for 4 hrs, the reaction mixture was poured into saturated aqueous sodium hydrogencarbonate, extracted with ethyl acetate, dried over magnesium sulfate, and evaporated in vacuo. The residue was purified by silica gel column chromatography eluting with n-hexane/ethyl acetate (1/1) to give 0.24 g of desired compound as an oil (56.4%).

IR (Neat, cm⁻¹): 3159, 3118, 3078, 3006, 2939, 2848, 1610.

NMR (DMSO-d₆, δ): 3.74(3H, s), 6.89(2H, dt, J=8.9 Hz and 1.9 Hz), 7.01(1H, t, J=54.8 Hz) 7.23(2H, dt, J=8.9 Hz and 2.0 Hz), 7.54-7.69(4H, m), 7.92(1H, t, J=2.3 Hz).

MS: 449 (M+H)⁺.

### EXAMPLE 34

### 1-(4-Cyanophenyl)-4-difluoromethyl-2-(4-methoxyphenyl)-1H-imidazole

A suspension of 4-difluoromethyl-2-(4-methoxyphenyl)-1-(4-trifluoromethanesulfonyloxyphenyl)-1H-imidazole obtained by Example 33 (0.2 g), zinc cyanide (Zn(CN)₂) (55 mg) and tetrakis(triphenylphosphine)palladium (Pd(PPh₃)₄) (272 mg) in N,N-dimethylformamide (1 ml) was stirred at 85° C. for overnight under nitrogen atmosphere then cooled to room temperature.

After filtration, the reaction mixture was poured into water, extracted with ethyl acetate, dried over magnesium sulfate, and evaporated in vacuo. The residue was purified by silica gel column chromatography eluting with n-hexane/ethyl acetate (1/1) to give 83 mg of desired compound (47.7%).

MP: 131-132° C.

IR (KBr, cm⁻¹): 3222, 3157, 3114, 2966, 2839, 2231, 1610.

NMR (DMSO-d₆, δ): 3.75(3H, s), 6.91(2H, dt, J=8.9 Hz and 1.9 Hz), 7.02(1H, t, J=54.8 Hz), 7.24(2H, dt, J=8.8 Hz and 2.0 Hz), 7.55 (2H, dt, J=8.7 Hz and 1.7 Hz), 7.93(1H, t, J=2.2 Hz), 7.95(2H, dt, J=8.5 Hz and 2.0 Hz).

MS: 326 (M+H)⁺.

### EXAMPLE 35-1

### 1-(4-Benzyloxyphenyl)-4,5-dihydro-4-ethoxycarbonyl-2-(2-methoxy-5-pyridinyl)-1H-imidazole

2.67 g of desired compound was obtained from a mixture of N¹-(4-benzyloxyphenyl)-2-methoxy-5-amidinopyridine (2.57 g) and ethyl 2-chloacrylate (1.56 g) in a manner similar to that of Example 12-1 (80.3%).

IR (Neat, cm⁻¹): 3448, 3411, 3378, 3037, 2981, 2949, 2902, 1734, 1608.

NMR (DMSO-d₆, δ): 1.24(3H, t, J=7.1 Hz), 3.83(3H, s), 4.06(2H, d, J=9.9 Hz), 4.17(2H, q, J=7.1 Hz), 4.81(1H, t, J=9.8 Hz), 5.04(2H, s), 6.77(1H, d, J=8.6 Hz), 6.93(4H, s), 7.29-7.44(5H, m), 7.68(1H, dd, J=8.6 Hz and 2.4 Hz), 8.18(1H, d, J=2.4 Hz).

MS: 432 (M+H)⁺.

### EXAMPLE 35-2

### 1-(4-Benzyloxyphenyl)-4-ethoxycarbonyl-2-(2-methoxy-5-pyridinyl)-1H-imidazole

1.74 g of desired compound was obtained from a suspension of 1-(4-benzyloxyphenyl)-4,5-dihydro-4-ethoxycarbonyl-2-(2-methoxy-5-pyridinyl)-1H-imidazole obtained by Example 35-1 (2.67 g) in N,N-dimethylformamide (27 ml) in a manner similar to that of Example 25-2 (65.5%).

MP: 109-110° C.

IR (KBr, cm⁻¹): 3433, 3390, 3136, 3070, 2976, 2941, 2841, 1693, 1608.

NMR (DMSO-d₆, δ): 1.29(3H, t, J=7.1 Hz), 3.84(3H, s), 4.28(2H, q, J=7.1 Hz), 5.15(2H, s), 6.80(1H, d, J=8.6 Hz), 7.12(2H, d, J=8.9 Hz), 7.32-7.49(7H, m), 7.65(1H, dd, J=8.6 Hz and 2.4 Hz), 8.06(1H, d, J=2.4 Hz), 8.12(1H, s).

MS: 430 (M+H)⁺.

### EXAMPLE 36

### 1-(4-Benzyloxyphenyl)-4-formyl-2-(2-methoxy-5-pyridinyl)-1H-imidazole

0.83 g of desired compound was obtained from 1-(4-benzyloxyphenyl)-4-ethoxycarbonyl-2-(2-methoxy-5-pyridinyl)-1H-imidazole (1.46 g) in a manner similar to that of Example 30 (63.3%).

IR (Neat, cm⁻¹): 3217, 3126, 3059, 2947, 2831, 2760, 1687, 1606.

NMR (DMSO-d₆, δ): 3.84(3H, s), 5.16(2H, s), 6.82(1H, d, J=8.5 Hz), 7.14(2H, dt, J=8.9 Hz and 2.0 Hz), 7.35-7.50(7H, m), 7.66(1H, dd, J=8.6 Hz and 2.5 Hz), 8.11(1H, d, 2.3 Hz), 8.35(1H, s), 9.84(1H, s).

MS: 386 (M+H)⁺.

### EXAMPLE 37

### 1-(4-Benzyloxyphenyl)-4-difluoromethyl-2-(2-methoxy-5-pyridinyl)-1H-imidazole

0.48 g of desired compound was obtained from 1-(4-benzyloxyphenyl)-4-formyl-2-(2-methoxy-5-pyridinyl)-1H-imidazole obtained by Example 36 (0.83 g) in a manner similar to that of Example 29-1 (54.7%).

IR (Neat, cm⁻¹): 3429, 3209, 3151, 3064, 3028, 2979, 2949, 2875, 2549, 1734, 1604.

NMR (DMSO-d₆, δ): 3.84(3H, s), 5.15(2H, s), 6.80(1H, d, J=8.5 Hz), 7.00(1H, t. J=54.8 Hz), 7.12(2H, d, J=9.0 Hz), 7.27-7.49(7H, m), 7.63(1H, dd, J=8.6 Hz and 2.5 Hz), 7.81(1H, t, J=2.2 Hz), 8.07(1H, d, J=1.8 Hz).

MS: 408 (M+H)⁺.

### EXAMPLE 38

### 4-Difluoromethyl-1-(4-hydoxyphenyl)-2-(2-methoxy-5-pyridinyl)-1H-imidazole

0.48 g of desired compound was obtained from 1-(4-benzyloxyphenyl)-4-difluoromethyl-2-(2-methoxy-5-pyridinyl)-1H-imidazole obtained by Example 37 (0.48 g) in a manner similar to that of Example 32 (ca.100%).

MP: 155-156° C.

IR (KBr, cm⁻¹): 3012, 2962, 2808, 2681, 2603, 1603.

NMR (DMSO-d₆, δ): 3.83(3H, s), 6.77-6.86(3H, m), 6.99(1H, t, J=54.9 Hz), 7.19(2H, d, J=8.8 Hz), 7.63(1H, dd, J=8.7 Hz and 2.5 Hz), 7.76(1H, t, J=2.2 Hz), 8.06(1H, d, J=2.4 Hz), 10.06(1H, br).

MS: 318 (M+H)⁺.

### EXAMPLE 39

### 4-Difluoromethyl-2-(2-methoxy-5-pyridinyl)-1-(4-trifluoromethanesulfonyloxyphenyl)-1H-imidazole

0.2 g of desired compound was obtained from 4-difluoromethyl-1-(4-hydroxyphenyl)-2-(2-methoxy-5-pyridinyl)-1H-imidazole obtained by Example 38 (0.17 g) in a manner similar to that of Example 33(83.1%).

IR (Neat, cm⁻¹): 3429, 3224, 3165, 3084, 3020, 2958, 2860, 1724, 1664, 1604.

NMR (DMSO-d₆, δ): 3.84(3H, s), 6.80(2H, d, J=8.4 Hz), 7.03(1H, t, J=54.8 Hz), 7.56-7.71(4H, m), 7.99(1H, t, J=2.2 Hz), 8.09(1H, d, J=2.4 Hz).

MS: 450 (M+H)⁺.

### EXAMPLE 40

### 1-(4-Cyanophenyl)-4-difluoromethyl-2-(2-methoxy-5-pyridinyl)-1H-imidazole

62 mg of desired compound was obtained from 4-difluoromethyl-2-(2-methoxy-5-pyridinyl)-1-(4-trifluoromethanesulfonyloxyphenyl)-1H-imidazole obtained by Example 39 (0.2 g) in a manner similar to that of Example 34 (42.7%).

MP: 160-161° C.

IR (KBr, cm⁻¹): 3219, 3140, 3101, 3051, 3005, 2985, 2954, 2241, 1608.

NMR (DMSO-d₆, δ): 3.85(3H, s), 6.82(1H, d, J=8.6 Hz), 7.04(1H, t, J=54.7 Hz), 7.57-7.63(3H, m), 7.99-8.03(3H, m), 8.11(1H, d, J=2.3 Hz).

MS: 327 (M+H)⁺.

### EXAMPLE 41-1

### 4-Ethoxycarbonyl-4,5-dihydro-1-(4-methoxyphenyl)-2-(2-methoxy-5-pyridinyl)-1H-imidazole

5.41 g of desired compound was obtained from a mixture of N¹-(4-methoxyphenyl)-2-methoxy-5-amidinopyridine (5 g) and ethyl 2-chloroacrylate (3.92 g) in a manner similar to that of Example 12-1 (78.3%).

IR (Neat, cm⁻¹): 3448, 3429, 3411, 3381, 3047, 2981, 2951, 2904, 2841, 1736, 1608.

NMR (DMSO-d₆, δ): 1.24(3H, t, J=7.1 Hz), 3.73(3H, s), 3.83(3H, s), 4.05(2H, d, J=9.5 Hz), 4.17(2H, q, J=7.1 Hz), 4.81(1H, t, J=9.5 Hz), 6.77(1H, d, J=8.5 Hz), 6.79-6.96(4H, m), 7.67(1H, dd, J=8.6 Hz and 2.4 Hz), 8.17(1H, d, J=2.3 Hz).

MS: 356 (M+H)⁺.

### EXAMPLE 41-2

### 4-Ethoxycarbonyl-1-(4-methoxyphenyl)-2-(2-methoxy-5-pyridinyl)-1H-imidazole

3.71 g of desired compound was obtained from a suspension of 4-ethoxycarbonyl-4,5-dihydro-1-(4-methoxyphenyl)-2-(2-methoxy-5-pyridinyl)-1H-imidazole obtained by Example 41-1 (5.41 g) in N,N-dimethylformamide (54 ml) in a manner similar to that of Example 25-2 (69%).

MP: 135-137° C.

IR (KBr, cm⁻¹): 3413, 3224, 3145, 3070, 2949, 2902, 2837, 1703, 1610.

NMR (DMSO-d₆, δ): 1.29(3H, t, J=7.1 Hz), 3.81(3H, s), 3.83(3H, s), 4.28(2H, q, J=7.1 Hz), 6.80(1H, d, J=8.6 Hz), 7.04(2H, dt, J=8.9 Hz and 2.0 Hz), 7.34(2H, dt, J=8.9 Hz and 2.2 Hz), 7.64(1H, dd, J=8.6 Hz and 2.5 Hz), 8.06(1H, d, J=2.4 Hz), 8.11(1H, s).

MS: 354 (M+H)⁺.

### EXAMPLE 42

### 4-Formyl-1-(4-methoxyphenyl)-2-(2-methoxy-5-pyridinyl)-1H-imidazole

0.88 g of desired compound was obtained from 4-ethoxycarbonyl-1-(4-methoxyphenyl)-2-(2-methoxy-5-pyridinyl)-1H-imidazole obtained by Example 41-2 (1.7 g) in a manner similar to that of Example 30 (59.1%).

IR (Neat, cm⁻¹): 3435, 3367, 3134, 3074, 3006, 2960, 2846, 1682, 1608.

NMR (DMSO-d₆, δ): 3.81(3H, s), 3.84(3H, s), 6.82(1H, d, J=8.5 Hz), 7.06(2H, dt, J=8.9 Hz and 1.9 Hz), 7.37(2H, dt, J=8.9 Hz and 1.9 Hz), 7.65(1H, dd, J=8.6 Hz and 2.5 Hz), 8.11(1H, d, J=2.1 Hz), 8.34(1H, s), 9.84(1H, s).

MS: 310 (M+H)⁺.

### EXAMPLE 43

### 4-Difluoromethyl-1-(4-methoxyphenyl)-2-(2-methoxy-5-pyridinyl)-1H-imidazole

332 mg of desired compound was obtained from 4-formyl-1-(4-methoxyphenyl)-2-(2-methoxy-5-pyridinyl)-1H-imidazole obtained by Example 42 (0.83 g) in a manner similar to that of Example 31 (36.5%).

MP: 106-107° C.

IR (KBr, cm⁻¹): 3398, 3153, 3114, 2997, 2947, 2844, 1606.

NMR (DMSO-d₆, δ): 3.81(3H, s), 3.83(3H, s), 6.80(1H, d, J=8.8 Hz), 7.00(1H, t, J=54.9 Hz), 7.04(2H, dt, J=8.9 Hz and 2.1 Hz), 7.33(2H, dt, J=8.8 Hz and 2.1 Hz), 7.63(1H, dd, J=8.6 Hz and 2.5 Hz), 7.80(1H, t, J=2.3 Hz), 8.07(1H, d, J=2.4 Hz).

MS: 332 (M+H)⁺.

### EXAMPLE 44

### 4-Ethoxycarbonyl-1-(4-methoxyphenyl)-2-(2-methoxy-5-pyridinyl)-1H-imidazole

1.5 g of desired compound was obtained from a suspension of N¹-(4-methoxyphenyl)-2-methoxy-5-amidinopyridine (1.5 g) in 2-propanol (10 ml) in a manner similar to that of Example 9 (72.8%).

### EXAMPLE 45

### 1-(4-Benzyloxyphenyl)-4-carboxy-2-(4-methoxyphenyl)-1H-imidazole

To a solution of 4-ethoxycarbonyl-1-(4-benzyloxyphenyl)-2-(4-methoxyphenyl)-1H-imidazole obtained by Example 29-2 (1.46 g) in ethanol (10 ml) and tetrahydrofuran (10 ml), 1N aqueous sodium hydroxide (6.81 ml) was added.

After stirring at room temperature overnight, the reaction mixture was poured into water and ethyl acetate, and extracted with water. Then, the water layer was acidified with 1N hydrochloric acid, extracted with ethyl acetate, dried over magnesium sulfate, and evaporated in vacuo. The resulting precipitates were collected by filtration and washed with diisopropyl ether to give the target compound (1.1 g).

MP: 113-115° C.

1H NMR (200 MHz, δ): 3.75(3H, s), 5.15(2H, s), 6.88(2H, d, J=8.8 Hz), 7.10(2H, d, J=8.9 Hz), 7.24-7.45(9H, m), 7.96(1H, s), 11.0-12.5(1H, br).

IR (KBr, cm⁻¹): 3392, 3224, 3145, 3076, 2972, 2935, 2893, 1701, 1610.

### EXAMPLE 46

### 1-(4-Benzyloxyphenyl)-4-(N-ethyl-N-methylcarbamoyl)-2-(4-methoxyphenyl)-1H-imidazole

A mixture of 1-(4-benzyloxyphenyl)-4-carboxy-2-(4-methoxyphenyl)-1H-imidazole obtained by Example 45 (0.44 g), ethylmethylamine (118 ml), 1-hydroxybenzotriazole (186 mg) and 1-(3-dimethylaminopropyl)-3-ethylcarbodiimide hydrochloride (263 mg) in N,N-dimethylformamide (5 ml) was stirred at room temperature overnight.

The reaction mixture was poured into water, extracted with ethyl acetate, dried over magnesium sulfate, and evaporated in vacuo. The residue was purified by silica-gel column chromatography eluting with (n-Hexane:Ethyl acetate=1:1). The resulting precipitates were corrected by filtration and washed with diisopropyl ether to give the target compound (0.44 g).

MP: 118-119° C.

1H NMR (DMSO-d₆, δ): 1.06-1.28(3H, m), 2.91-3.02(2H, m), 3.40-3.54(2H, m), 3.74(3H, s), 3.93-4.07(1H, m), 5.15(2H, s), 6.88(2H, d, J=8.8 Hz), 7.10(2H, d, J=8.9 Hz), 7.24-7.30(4H, m), 7.36-7.49(5H, m), 7.73(1H, s).

IR (KBr, cm⁻¹): 3124, 3066, 2958, 2935, 2839, 1608.

Mass m/e: 442 (M⁺+1).

### EXAMPLE 47

### 4-(N-Ethyl-N-methylcarbamoyl)-1-(4-hydroxyphenyl)-2-(4-methoxyphenyl)-1H-imidazole

The target compound was obtained from 1-(4-benzyloxyphenyl)-4-(N-ethyl-N-methylcarbamoyl)-2-(4-methoxyphenyl)-1H-imidazole obtained by Example 46 in a manner similar to that of Example 62 described later.

1H NMR (DMSO-d₆, δ): 1.10-1.28(3H, m), 2.90-3.02(2H, m), 3.40-3.50(2H, m), 3.74(3H, s), 3.91-4.03(1H, m), 6.82(2H, d, J=8.7 Hz), 6.88(2H, d, J=8.9 Hz), 7.11(1H, s), 7.14(2H, d, J=8.7 Hz), 7.27(2H, d, J=8.7 Hz), 7.67(1H, s).

IR (KBr, cm⁻¹): 3126, 3091, 3018, 2968, 2933, 2831, 2738, 2677, 2600, 2476, 1612.

MS m/e: 352 (M⁺+1).

### EXAMPLE 48

### 1-(4-Benzyloxyphenyl)-4-(N,N-diethylcarbamoyl)-2-(4-methoxyphenyl)-1H-imidazole

The target compound was obtained from 1-(4-benzyloxyphenyl)-4-carboxy-2-(4-methoxyphenyl)-1H-imidazole obtained by Example 45 and N,N-diethylamine in a manner similar to that of Example 46.

MP: 146-147° C.

1H NMR (DMSO-d₆, δ): 1.10-1.30(6H, m), 3.38-3.50(2H, m), 3.74(3H, s), 3.85-4.02(2H, m), 5.15(2H, s), 6.88(2H, d, J=8.8 Hz), 7.10(2H, d, J=8.9 Hz), 7.24-7.30(4H, m), 7.36-7.49(5H, m), 7.72(1H, s).

IR (KBr, cm⁻¹): 3113, 2972, 2929, 1593.

MS m/e: 456 (M⁺+1).

### EXAMPLE 49

### 4-(N,N-Diethylcarbamoyl)-1-(4-hydroxyphenyl)-2-(4-methoxyphenyl)-1H-imidazole

The target compound was obtained from 1-(4-benzyloxyphenyl)-4-(N,N-diethylcarbamoyl)-2-(4-methoxyphenyl)-1H-imidazole obtained by Example 48 in a manner similar to that of Example 62 described later.

1H NMR (DMSO-d₆, δ): 1.02-1.30(6H, m), 3.22-3.48(2H, m), 3.73(3H, s), 3.83-4.02(2H, m), 6.81-6.92(4H, m), 7.14(2H, dd, J=6.7 Hz, 2.0 Hz), 7.27(2H, dt, J=9.4 Hz, 2.5 Hz), 7.66(1H, s).

IR (KBr, cm⁻¹): 3145, 3030, 2970, 2937, 2833, 1693, 1606.

MS m/e: 366 (M⁺+1).

### EXAMPLE 50

### 1-(4-Benzyloxyphenyl)-2-(4-methoxyphenyl)-4-(1-piperidinecarbonyl)-1H-imidazole

The target compound (0.5 g) was obtained from 1-(4-benzyloxyphenyl)-4-carboxy-2-(4-methoxyphenyl)-1H-imidazole obtained by Example 45 and piperidine in a manner similar to that of Example 46.

1H NMR (200 MHz, δ): 1.507-1.572(4H, m), 1.605-1.67(2H, m), 3.462-3.644(2H, m), 3.74(3H, s), 3.918-4.244(2H, m), 5.144(2H, s), 6.879(2H, d, J=4.5 Hz), 7.096(2H, d, J=4.5 Hz), 7.251(2H, d, J=4.3 Hz), 7.278(2H, d, J=4.3 Hz), 7.348-7.478(5H, m), 7.721(1H, s).

IR (KBr, cm⁻¹): 3116, 3033, 2931, 2850.

MS m/e: 468 (M+H)⁺.

### EXAMPLE 51

### 1-(4-Hydroxyphenyl)-2-(4-methoxyphenyl)-4-(1-piperidinecarbonyl)-1H-imidazole

The target compound (0.41 g) was obtained from 1-(4-benzyloxyphenyl)-2-(4-methoxyphenyl)-4-(1-piperidinecarbonyl)-1H-imidazole obtained by Example 50 in a manner similar to that of Example 62 described later.

1H NMR (200 MHz, δ): 1.509-1.577(4H, m), 1.611-1.674(2H, m), 3.51-3.657(2H, m), 3.734(3H, s), 4.035-4.224(2H, m), 6.814(2H, d, J=4.4 Hz), 6.881(2H, d, J=4.3 Hz), 7.136(2H, d, J=4.4 Hz), 7.256(2H, d, J=4.4 Hz), 7.668(1H, s), 9.908(1H, bs).

IR (KBr, cm⁻¹): 3151, 3035, 2935, 2852, 1606.

MS m/e: 378 (M+H)⁺.

### EXAMPLE 52-1

### N¹-(4-Benzyloxyphenyl)-4-methoxybenzamidine

To a solution of 4-benzyloxyaniline hydrochloride (3 g) in tetrahydrofuran (15 ml), 1.0 M sodium bis(trimethylsilyl)amide in tetrahydrofuran (26.7 ml) was added dropwise at room temperature. After the mixture was stirred for 20 min, anisonitrile (1.69 g) was added.

The reaction mixture was stirred for 4 hrs, and then poured into 300 ml of ice-water. The precipitates were collected by filtration, washed with diisopropyl ether to give the target compound (3.3 g).

1H NMR (200 MHz, δ): 3.8(3H, s), 5.05(2H, s), 6.09(2H, bs), 6.74-6.8(2H, m), 6.96(4H, d, J=8.5 Hz), 7.29-7.49(5H, m), 7.92(2H, d, J=8.9 Hz).

MS m/e: 333 (M+H)⁺.

### EXAMPLE 52-2

### 4-Cyano-4,5-dihydro-1-(4-benzyloxyphenyl)-2-(4-methoxyphenyl)-1H-imidazole

A mixture of N¹-(4-Benzyloxyphenyl)-4-methoxybenzamidine obtained by Example 52-1 (2 g), 2-chlorocyanoethylene (0.36 ml) and N,N-diisopropylethylamine (0.79 ml) in tetrahydrofuran (10 ml) was stirred at reflux condition overnight.

After cooling to room temperature, the reaction mixture was poured into water, and extracted with ethyl acetate. The organic layer was washed with water and brine, then dried over magnesium sulfate and evaporated in vacuo. The residue was purified by silica-gel column chromatography eluting with (n-Hexane:Ethyl acetate=1:1) to give the target compound (0.82 g).

MP: 121-122° C.

1H NMR (200 MHz, δ): 3.74(3H, s), 4.11-4.19(2H, m), 5.03(2H, s), 5.16-5.25(1H, m), 6.87(2H, d, J=9 Hz), 6.93(4H, s), 7.29-7.44(7H, m).

MS (ESI⁺) m/e: 384 (M+H)⁺.

### EXAMPLE 52-3

### 4-Cyano-1-(4-benzyloxyphenyl)-2-(4-methoxyphenyl)-1H-imidazole

A suspension of 4-cyano-4,5-dihydro-1-(4-benzyloxyphenyl)-2-(4-methoxyphenyl)-1H-imidazole obtained by Example 52-2 (0.8 g) and manganese(IV) oxide (0.91 g) in N,N-dimethylformamide (8 ml) was stirred at 100° C. for 4 hrs.

After filtration, the reaction mixture was poured into water, extracted with ethyl acetate, dried over magnesium sulfate, and evaporated in vacuo. To the solution of the residue in N,N-dimethylformamide (8 ml), phosphorus oxychloride (0.58 ml) was added under stirring at 0° C.

After stirring at room temperature for 2 hrs, the reaction mixture was poured into saturated aqueous sodium hydrogencarbonate, extracted with ethyl acetate, dried over magnesium sulfate, and evaporated in vacuo. The residue was purified by silica-gel column chromatography eluting with (n-Hexane:Ethyl acetate=3:1 to 1:1) to give the target compound (0.74 g) as an oil.

1H NMR (200 MHz, δ): 3.75(3H, s), 5.16(2H, s), 6.89(2H, d, J=8.5 Hz), 7.12(2H, d, J=9 Hz), 7.25-7.48(9H, m), 8.4(1H, s).

MS (ESI⁺) m/e: 382 (M+H)⁺.

### EXAMPLE 53

### 4-Cyano-1-(4-hydroxyphenyl)-2-(4-methoxyphenyl)-1H-imidazole

The target compound was obtained from 4-cyano-1-(4-benzyloxyphenyl)-2-(4-methoxyphenyl)-1H-imidazole obtained by Example 52-3 in a manner similar to that of Example 62 described later.

1H NMR (CDCl₃, δ): 3.74(3H, s), 6.75-6.95(4H, m), 7.10-7.35(4H, m), 8.36(1H, s), 9.98(1H, bs).

MS (ESI, m/e): 292 (M+1).

### EXAMPLE 54-1

### 1-(4-Benzyloxyphenyl)-4-cyano-4,5-dihydro-2-(2-methoxy-5-pyridinyl)-1H-imidazole

The target compound was obtained from N¹-(4-Benzyloxyphenyl)-2-methoxy-5-pyridinyl amidine in a manner similar to that of Example 52-2.

1H NMR (200 MHz, δ): 3.84(3H, s), 4.15-4.21(2H, m), 5.05(2H, s), 5.25(1H, dd, J=8.8, 10.5 Hz), 6.78(1H, d, J=8.5 Hz), 6.92-7.04(4H, m), 7.32-7.45(5H, m), 7.66(1H, dd, J=2.5, 8.5 Hz), 8.19(1H, d, J=2 Hz).

MS (ESI⁺) m/e: 385 (M+H)⁺.

### EXAMPLE 54-2

### 1-(4-Benzyloxyphenyl)-4-cyano-2-(2-methoxy-5-pyridinyl)-1H-imidazole

The target compound was obtained from 1-(4-Benzyloxyphenyl)-4-cyano-4,5-dihydro-2-(2-methoxy-5-pyridinyl)-1H-imidazole obtained by Example 54-1 in a manner similar to that of Example 52-3.

1H NMR (200 MHz, δ): 3.84(3H, s), 5.16(2H, s), 6.81(1H, d, J=8 Hz), 7.14(2H, d, J=9 Hz), 7.316-7.5(7H, m), 7.63(1H, dd, J=2.3, 8.5 Hz), 8.1(1H, dd, J=2.5 Hz), 8.47(1H, s).

MS (ESI⁺) m/e: 383 (M+H)⁺.

### EXAMPLE 55

### 1-(4-Benzyloxyphenyl)-4-ethylcarbonyl-2-(2-methoxy-5-pyridinyl)-1H-imidazole

To a solution of 1-(4-benzyloxyphenyl)-4-cyano-2-(2-methoxy-5-pyridinyl)-1H-imidazole obtained by Example 54-2 (1.1 g) in tetrahydrofuran (10 ml), 1N solution of ethylmagunesium bromide in tetrahydrofuran (8.63 ml) was added under stirring at 0° C.

After stirring at room temperature for 1 hr, the reaction mixture was poured into aqueous 10% potassium hydrogen sulfate and stirred at room temperature for 30 min. The mixture was alkalinized with saturated aqueous sodium hydrogen carbonate, extracted with ethyl acetate, washed with water, dried over magnesium sulfate, and evaporated in vacuo. The resulting precipitates were collected by filtration and washed with diisopropyl ether to give the target compound (1.07 g).

MP: 126-128° C.

1H NMR (DMSO-d₆, δ): 1.10(3H, t, J=7.4 Hz), 2.95(2H, q, J=7.4 Hz), 3.84(3H, s), 5.16(2H, s), 6.81(1H, d, J=8.6 Hz), 7.12(2H, d, J=8.9 Hz), 7.32-7.49(7H, m), 7.66(1H, dd, J=8.6 Hz, 2.4 Hz), 8.08(1H, d, J=2.4 Hz), 8.17(1H, s).

IR (KBr, cm⁻¹): 3217, 3126, 3066, 3030, 2972, 2939, 2883, 1666, 1610.

MS m/e: 414 (M⁺+1).

### EXAMPLE 56

### 4-Ethylcarbonyl-1-(4-hydroxyphenyl)-2-(2-methoxy-5-pyridinyl)-1H-imidazole

The target compound was obtained from 1-(4-benzyloxyphenyl)-4-ethylcarbonyl-2-(2-methoxy-5-pyridinyl)-1H-imidazole obtained by Example 55 in a manner similar to that of Example 62 described later.

MP: 221-223° C.

1H NMR (DMSO-d₆, δ): 1.10(3H, t, J=7.3 Hz), 2.95(2H, q, J=7.3 Hz), 3.84(3H, s), 6.79-6.88(3H, m), 7.20(2H, dt, J=9.6 Hz, 2.7 Hz), 7.66(1H, dd, J=8.7 Hz, 2.4 Hz), 8.07(1H, d, J=2.4 Hz), 9.97(1H, s).

IR (KBr, cm⁻¹): 3215, 3136, 3053, 2978, 2947, 2900, 1676, 1603.

MS m/e: 324 (M⁺+1).

### EXAMPLE 57

### 1-(4-Benzyloxyphenyl)-4-isopropylcarbonyl-2-(2-methoxy-5-pyridinyl)-1H-imidazole

The target compound (1.04 g) was obtained from 1-(4-benzyloxyphenyl)-4-cyano-2-(2-methoxy-5-pyridinyl)-1H-imidazole obtained by Example 54-2 in a manner similar to that of Example 55.

MP: 118-120° C.

1H NMR (DMSO-d₆, δ): 1.14(6H, d, J=6.8 Hz), 3.56-3.70(1H, m), 3.84(3H, s), 5.16(2H, s), 6.81(1H, d, J=8.5 Hz), 7.13(2H, dd, J=9.1 Hz, 2.3 Hz), 7.32-7.49(7H, m), 7.67(1H, dd, J=8.5 Hz, 2.4 Hz), 8.08(1H, d, J=2.4 Hz), 8.19(1H, s).

IR (KBr, cm⁻¹): 3126, 3064, 3033, 2968, 2875, 1660, 1608.

MS m/e: 428 (M⁺+1).

### EXAMPLE 58

### 4-Isopropylcarbonyl-1-(4-hydroxyphenyl)-2-(2-methoxy-5-pyridinyl)-1H-imidazole

The target compound was obtained from 1-(4-benzyloxyphenyl)-4-isopropylcarbonyl-2-(2-methoxy-5-pyridinyl)-1H-imidazole obtained by Example 57 in a manner similar to that of Example 62 described later.

MP: 185-187° C.

1H NMR (DMSO-d₆, δ): 1.14(6H, d, J=6.8 Hz), 3.56-3.69(1H, m), 3.84(3H, s), 6.79-6.86(3H, m), 7.17-7.25(2H, m), 7.67(1H, dd, J=8.8 Hz, 2.4 Hz), 8.07(1H, d, J=2.4 Hz), 8.14(1H, s), 9.98(1H, s).

IR (KBr, cm⁻¹): 3134, 2972, 2891, 2812, 2744, 2681, 2607, 1676, 1612.

MS m/e: 338 (M⁺+1).

### EXAMPLE 59

### 1-(4-Benzyloxyphenyl)-4-cyclopentylcarbonyl-2-(2-methoxy-5-pyridinyl)-1H-imidazole

To a solution of 1-(4-benzyloxyphenyl)-4-cyano-2-(2-methoxy-5-pyridinyl)-1H-imidazole obtained by Example 54-2 (0.8 g) in tetrahydrofuran (8 ml), 2N solution of cyclopentylmagnesium chloride in tetrahydrofuran (3.14 ml) was added under stirring at 0° C.

After stirring at room temperature for 2 hrs, the reaction mixture was poured into aqueous 10% potassium hydrogen sulfate and stirred at room temperature for 30 min. The mixture was alkalinized with saturated aqueous sodium hydrogen carbonate, extracted with ethyl acetate, washed with water, dried over magnesium sulfate, and evaporated in vacuo. The resulting precipitates were collected by filtration and washed with diisopropyl ether to give the target compound (0.82 g).

1H NMR (200 MHz, δ): 1.57-1.949(m, 8H), 3.764(1H, t, J=7.9 Hz), 3.84(3H, s), 5.156(2H, s), 6.81(1H, d, J=8.5 Hz), 7.12(2H, d, J=9 Hz), 7.328-7.501(7H, m), 7.669(1H, dd, J=8.5 Hz, 2.5 Hz), 8.078(1H, d, J=1 Hz), 8.188(1H, s).

IR (KBr, cm⁻¹): 3122, 2947, 2868, 1658, 1608.

MS (ESI⁺, m/e): 454 (M+H).

### EXAMPLE 60

### 4-Cyclopentylcarbonyl-1-(4-hydroxyphenyl)-2-(2-methoxy-5-pyridinyl)-1H-imidazole

The target compound was obtained from 1-(4-Benzyloxyphenyl)-4-cyclopentylcarbonyl-2-(2-methoxy-5-pyridinyl)-1H-imidazole obtained by Example 59 in a manner similar to that of Example 62 described later.

1H NMR (200 MHz, δ): 1.577-1.968(8H, m), 3.761(1H, t, J=8 Hz), 3.836(3H, s), 6.793-6.859(3H, m), 7.21(2H, d, J=7 Hz), 7.667(1H, dd, J=9 Hz, 2.5 Hz), 8.069(1H, d, J=1.5 Hz), 8.143(1H, s).

IR (KBr, cm⁻¹): 3220, 3124, 2960, 1674, 1608.

MS (ESI⁺, m/e): 364 (M+H).

### EXAMPLE 61

### 1-(4-Benzyloxyphenyl)-2-(4-methoxyphenyl)-4-trifluoromethyl-1H-imidazole

A mixture of N¹-(4-Benzyloxyphenyl)-4-methoxybenzamidine (1 g), 3-bromo-1,1,1-trifluoropropanone (0.47 ml) and sodium hydrogencarbonate (506 mg) in isopropyl alcohol (10 ml) was stirred at reflux condition overnight.

After cooling to room temperature, the reaction mixture was filtrated and evaporated in vacuo. Then the residue was poured into water, extracted with ethyl acetate, dried over magnesium sulfate, and evaporated in vacuo. The residue was purified by silica-gel column chromatography eluting with (n-Hexane:Ethyl acetate=1:1) to give the target compound (0.55 g) as an oil.

1H NMR (DMSO-d₆, δ): 3.75(3H, s), 5.16(2H, s), 6.86-6.92(2H, m), 7.09-7.13(2H, m), 7.25-7.50(9H, m), 8.08(1H, d, J=1.4 Hz).

IR (Neat, cm⁻¹): 3120, 3068, 2973, 2843, 1610.

MS m/e: 425 (M⁺+1).

### EXAMPLE 62

### 1-(4-Hydroxyphenyl)-2-(4-methoxyphenyl)-4-trifluoromethyl-1H-imidazole

1-(4-Benzyloxyphenyl)-2-(4-methoxyphenyl)-4-trifluoromethyl-1H-imidazole obtained by Example 61 (0.55 g) and dry 20% Pd(OH)₂/C (200 mg) in ethanol (10 ml) and cyclohexene (5 ml) was stirred at reflux condition for 2 hrs and cooled to room temperature.

After filtration, the reaction mixture was evaporated in vacuo to give the target compound (0.44 g).

MP: 215-216° C.

1H NMR (DMSO-d₆, δ): 3.74(3H, s), 6.81-6.92(4H, m), 7.16-7.30(4H, m), 8.03(1H, d, J=1.3 Hz).

IR (KBr, cm⁻¹): 3149, 3103, 3037, 2964, 2910, 2829, 2690, 2611, 1649, 1614.

MS m/e: 335 (M⁺+1).

[A] Analgesic Activity:

Effect on adjuvant arthritis in rats:

(i) Test Method:

Analgesic activity of a single dose of agents in arthritic rats was studied.

Arthritis was induced by injection of 0.5 mg of Mycobacterium tuberculosis (Difco Laboratories, Detroit, Mich.) in 50 μl of liquid paraffin into the right hind footpad of Lewis rats aged 7 weeks. Arthritic rats were randomized and grouped (n=10) for drug treatment based on pain threshold of left hind paws and body weight on day 22.

Drugs (Test compounds) were administered and the pain threshold was measured 2 hrs after drug administration. The intensity of hyperalgesia was assessed by the method of Randall—Selitto. The mechanical pain threshold of the left hind paw (uninjected hind paw) was determined by compressing the ankle joint with a balance pressure apparatus (Ugo Basile Co. Ltd., Varese, Italy). The threshold pressure of rats squeaking or struggling was expressed in grams. The threshold pressure of rats treated with drugs was compared with that of non-treated rats. A dose showing the ratio of 1.5 is considered to be the effective dose.

(ii) Test Results:

| Test compound | Dose | The coefficient |
|---|---|---|
| (Example No.) | (mg/kg) | of analgesic |
| 3-2 | 3.2 | >1.5 |
| 11 | 3.2 | >1.5 |
| 24 | 3.2 | >1.5 |
| 28 | 3.2 | >1.5 |
| 40 | 3.2 | >1.5 |
| 43 | 3.2 | >1.5 |

[B] Inhibiting Activity Against COX-I and COX-II
(Whole Blood Assay):
(i) Test Method:
Whole Blood Assay for COX-I

Fresh blood was collected by syringe without anticoagulants from volunteers with consent. The subjects had no apparent inflammatory conditions and had not taken any medication for at least 7 days prior to blood collection.

500 μl aliquots of human whole blood were immediately incubated with 2 μl of either dimethyl sulfoxide vehicle or a test compound at final concentrations for 1 hr at 37° C. to allow the blood to clot. Appropriate treatments (no incubation) were used as blanks. At the end of the incubation, 5 μl of 250 mM Indomethacin was added to stop the reaction. The blood was centrifuged at 6000×g for 5 min at 4° C. to obtain serum. A 100 μl aliquot of serum was mixed with 400 μl methanol for protein precipitation. The supernatant was obtained by centrifuging at 6000×g for 5 min at 4° C. and was assayed for TXB₂ using an enzyme immunoassay kit according to the manufacturer's procedure. For a test compound, the results were expressed as percent inhibition of thromboxane B₂(TXB₂) production relative to control incubations containing dimethyl sulfoxide vehicle.

The data were analyzed by that a test compound at the indicated concentrations was changed log value and was applied simple linear regression. IC₅₀ value was calculated by least squares method.

Whole Blood Assay for COX-II

Fresh blood was collected in heparinized tubes by syringe from volunteers with consent. The subjects had no apparent inflammatory conditions and had not taken any medication for at least 7 days prior to blood collection.

500 μl aliquots of human whole blood were incubated with either 2 μl dimethyl sulfoxide vehicle or 2 μl of a test compound at final concentrations for 15 min at 37° C. This was followed by incubation of the blood with 101 of 5 mg/ml lipopolysaccharide for 24 hrs at 37° C. for induction of COX-II. Appropriate PBS treatments (no LPS) were used as blanks. At the end of the incubation, the blood was centrifuged at 6000×g for 5 min at 4° C. to obtain plasma. A 100 μl aliquot of plasma was mixed with 400 μl methanol for protein precipitation. The supernatant was obtained by centrifuging at 6000×g for 5 min at 4° C. and was assayed for prostaglandin E₂ (PGE₂) using a radioimmunoassay kit after conversion of PGE₂ to its methyl oximate derivative according to the manufacturer's procedure.

For a test compound, the results were expressed as percent inhibition of PGE₁ production relative to control incubations containing dimethyl sulfoxide vehicle. The data were analyzed by that a test compound at the indicated concentrations was changed log value and was applied simple linear regression. IC₅₀ value was calculated by least squares method.

(ii) Test Results:

| Test Compound | COX-I | COX-II |
|---|---|---|
| (Example No.) | IC50 (μM) | IC50 (μM) |
| 1-2 | <0.01 | ≧0.1 |
| 3-2 | <0.01 | ≧0.1 |
| 4-2 | <0.01 | ≧0.1 |
| 8 | <0.01 | ≧0.1 |
| 11 | <0.01 | ≧0.1 |
| 17 | <0.01 | ≧0.1 |
| 20 | <0.01 | ≧0.1 |
| 21 | <0.01 | ≧0.1 |
| 24 | <0.01 | ≧0.1 |
| 34 | <0.01 | ≧0.1 |
| 40 | <0.01 | ≧0.1 |
| 43 | <0.01 | ≧0.1 |

It appeared, from the above-mentioned Test Results, that the compound (I) or pharmaceutically acceptable salts thereof of the present invention have an inhibiting activity against COX, particularly a selective inhibiting activity against COX-I.

Additionally, it was further confirmed that the compounds (I) of the present invention lack undesired side-effects of non-selective NSAIDs, such as gastrointestinal disorders, bleeding, renal toxicity, cardiovascular affection, or the like. Therefore, compound (I) or a salt thereof is expected to be useful as medicament.

The compound (I) and pharmaceutically acceptable salts thereof of this invention possess COX inhibiting activity and possesses strong anti-inflammatory, antipyretic, analgesic, antithrombotic, anti-cancer activities, and so on.

The compound (I) and pharmaceutically acceptable salt thereof, therefore, are useful for treating and/or preventing COX mediated diseases, inflammatory conditions, various pains, collagen diseases, autoimmune diseases, various immunological diseases, thrombosis, cancer and neurodegenerative diseases in human beings or animals by using administered systemically or topically.

More particularly, the object compound (I) and pharmaceutically acceptable salts thereof are useful for treating and/or preventing inflammation and acute or chronic pain in joint and muscle [e.g. rheumatoid arthritis, rheumatoid spondylitis, osteoarthritis, gouty arthritis, juvenile arthritis, or the like.], inflammatory skin condition [e.g. sunburn, burns, eczema, dermatitis, or the like.], inflammatory eye condition [e.g. conjunctivitis, or the like.], lung disorder in which inflammation is involved [e.g. asthma, bronchitis, pigeon fancier's disease, farmer's lung, or the like.], condition of the gastrointestinal tract associated with inflammation [e.g. aphthous ulcer, Chrohn's disease, atopic gastritis, gastritis varialoforme, ulcerative colitis, coeliac disease, regional ileitis, irritable bowel syndrome, or the like.], gingivitis, inflammation, pain and tumescence after operation or injury, pyrexia, pain and other conditions associated with inflammation, particularly those in which lipoxygenase and cyclooxygenase products are a factor, systemic lupus erythematosus, scleroderma, polymyositis, tendinitis, bursitis, periarteritis nodose, rheumatic fever, Sjogren's syndrome, Behcet disease, thyroiditis, type I diabetes, nephrotic syndrome, aplastic anemia, myasthenia gravis, uveitis contact dermatitis, psoriasis, Kawasaki disease, sarcoidosis, Hodgkin's disease, Alzheimers disease, or the like.

Additionally, the object compound (I) and salt thereof are expected to be useful as therapeutical and/or preventive agents for cardiovascular or cerebrovascular diseases, the diseases caused by hyperglycemia and hyperlipemia.

Further, compound (I) and salt thereof are expected to be useful as analgesic agent, which is usable for treating or preventing pains caused by or associated with acute or chronic inflammations, for example rheumatoid arthritis, osteoarthritis, lumbar rheumatism, rheumatoid spondylitis, gouty arthritis, juvenile arthritis; lumbago; cervico-omo-brachial syndrome; scapulohumeral periarthritis; pain and tumescence after operation or injury.

The patents, patent applications and publications cited herein are incorporated by reference.

This application is based on Australian Provisional Application No.2003902208 filed on May 8, 2003, Australian Provisional Application No.2003903861 filed on Jul. 24, 2003 and Australian Provisional Application No.2003904068 filed on Aug. 1, 2003, the contents of which are hereby incorporated by references.

1. A compound of the formula (I): 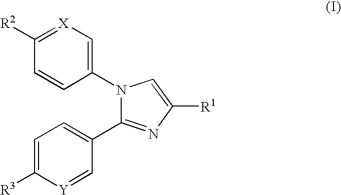
[wherein
R¹ is (lower)alkyl, halogen-substituted (lower)alkyl, hydroxy-substituted (lower)alkyl, cycloalkyl, carbamoyl, N-[(lower)alkyl]carbamoyl, N,N-di[(lower)alkyl]carbamoyl, formyl, (lower)alkanoyl, carboxy, [(lower)alkoxy]carbonyl, cyano, cycloalkylcarbonyl or heterocycliccarbonyl;
R² is halogen, cyano, hydroxy, (lower)alkoxy, aryl[(lower)alkyl]oxy, [(lower)alkoxy]carbonyl, carbamoyl, formyloxy, (lower)alkanoyloxy, [(lower)alkyl]sulfonyloxy, [halogen-substituted (lower)alkyl]sulfonyloxy or carboxy;
R³ is (lower)alkoxy, hydroxy, amino, [(lower)alkyl]amino, or di[(lower)alkyl]amino, or di [(lower)alkyl]amino;
X and Y are each CH or N]
or pharmaceutically acceptable salts thereof.

2. The compound of claim 1,
wherein
R¹ is(lower)alkyl, halogen-substituted(lower)alkyl, cycloalkyl, N,N-di[(lower)alkyl]carbamoyl, (lower)alkanoyl, or cyano;
R² is halogen, cyano, hydroxy, or lower alkoxy;
R³ is lower alkoxy; and
X and Y are each CH, X is N and Y is CH, or X is CH and Y is N;
or pharmaceutically acceptable salts thereof.

3. (canceled)

4. A composition comprising the compound of claim 1 as an active ingredient and a pharmaceutically acceptable carrier, excipient, or combination thereof.

5. A method for treating at least one disease in human beings or animals comprising administering the compound of claim 1 to the human beings or animals in an amount sufficient to treat the at least one disease.

6-7. (canceled)

8. The method of claim 5, wherein the at least one disease comprises rheumatoid arthritis, osteoarthritis, lumbar rheumatism, rheumatoid spondylitis, gouty arthritis, juvenile arthritis; lumbago; cervico-omo-brachial syndrome; scapulohumeral periarthritis; pain and tumescence after operation or injury.

9. (canceled)

10. The method of claim 5, wherein the at least one disease comprises inflammatory conditions, collagen diseases, autoimmune diseases, immunity diseases, thrombosis, cancer and neurodegerative diseases.

11. A composition comprising the compound of claim 2 as an active ingredient and a pharmaceutically acceptable carrier, excipient, or combination thereof.

12. A method for treating at least one disease in human beings or animals comprising administering the compound of claim 2 to the human beings or animals in an amount sufficient to treat the at least one disease.

13. The method of claim 12, wherein the at least one disease comprises rheumatoid arthritis, osteoarthritis, lumbar rheumatism, rheumatoid spondylitis, gouty arthritis, juvenile arthritis; lumbago; cervico-omo-brachial syndrome; scapulohumeral periarthritis; pain and tumescence after operation or injury.

14. The method of claim 12, wherein the at least one disease comprises inflammatory conditions, collagen diseases, autoimmune diseases, immunity diseases, thrombosis, cancer and neurodegerative diseases.

15. The compound of claim 1, wherein the pharmaceutically acceptable salts comprise alkali metal salts, alkaline earth metal salts, ammonium salts, organic base salts, organic acid salts, inorganic acid salts, and amino acid salts.

16. The compound of claim 2, wherein the pharmaceutically acceptable salts comprise alkali metal salts, alkaline earth metal salts, ammonium salts, organic base salts, organic acid salts, inorganic acid salts, and amino acid salts.

17. An intermediate of formula (II) 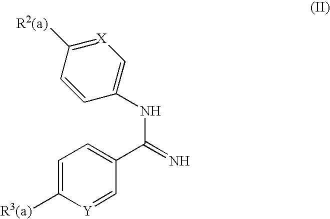
wherein R²(a) is selected from the group consisting of halogen, cyano, (lower)alkoxy, aryl((lower)alkyl)oxy, ((lower)alkoxy)carbonyl, formyloxy, (lower)alkanoyloxy, ((lower)alkyl)sulfonyloxy, and (halogen-substituted(lower)alkyl)sulfonyloxy;
wherein R³(a) is lower alkoxy; and
wherein
X and Y are each CH or N.

18. An intermediate of formula (III) 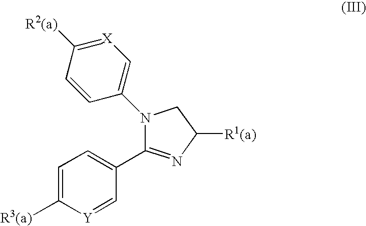
wherein R¹(a) is selected from the group consisting of (lower alkyl), halogen-substituted (lower)alkyl, cycloaklyl, N,N-di((lower)alkyl)carbamoyl, formyl, (lower)alkanol, ((lower)alkoxy)carbonyl, cyano and cycloalkylcarbonyl;
wherein R²(a) is selected from the group consisting of halogen, cyano, (lower)alkoxy, aryl((lower)alkyl)oxy, ((lower)alkoxy)carbonyl, formyloxy, (lower)alkanoyloxy, ((lower)alkyl)sulfonyloxy, and (halogen-substituted(lower)alkyl)sulfonyloxy;
wherein R³(a) is lower alkoxy; and
wherein
X and Y are each CH or N.

19. A package comprising
the compound of claim 1; and
a written matter associated therewith,
wherein the written matter states that the compound is used for preventing or treating at least one of inflammatory conditions, various pains, collagen diseases, autoimmune diseases, various immunity diseases, analgesic, thrombosis, cancer and neurodegerative diseases.

20. A package comprising
the compound of claim 2; and
a written matter associated therewith,
wherein the written matter states that the compound is used for preventing or treating at least one of inflammatory conditions, various pains, collagen diseases, autoimmune diseases, various immunity diseases, analgesic, thrombosis, cancer or neurodegerative diseases.
